(19) United States
(12) Patent Application Publication
(10) Pub. No.: US 2010/0203068 A1
(43) Pub. Date: Aug. 12, 2010

(54) METHODS OF SWITCHING THE PHENOTYPE OF T CELLS BY TRANSGENIC LINEAGE FACTOR FOXP3

(75) Inventors: Alexander G. Betz, Cambridge (DE); Kristian G. Andersen, Cambridge (DK)

Correspondence Address: NIXON PEABODY, LLP, 401 9TH STREET, NW, SUITE 900, WASHINGTON, DC, 20004-2128, US

(73) Assignee: LABORATORY OF MOLECULAR BIOLOGY, Cambridge (GB)

(21) Appl. No.: 12/678,724

(22) PCT Filed: Sep. 17, 2008
(86) PCT No.: PCT/GB2008/003143

§ 371 Date: Mar. 17, 2010

(30) Foreign Application Priority Data
Sep. 18, 2007 (GB) 0718160.5

(51) Int. Cl. A61K 39/00; C12N 15/63; C12N 5/10

(52) U.S. Cl. 424/184.1; 435/455; 435/325

(57) ABSTRACT

In one aspect the invention relates to a method of switching the phenotype of a target cell, said method comprising inducing lineage factor activity in said cell via a transgene. In another aspect, the invention relates to a method of switching the phenotype of a target cell, said method comprising introducing to said cell a genetic element capable of inducibly generating lineage factor activity, and inducing lineage factor activity in said cell. The invention also relates to methods of suppressing immune responses and methods of treating subjects.

### FIELD OF THE INVENTION

The invention relates to methods for inducing cell type switching, particularly switching of immune cell types. Specifically, the invention relates to methods of switching cell types by induction of lineage factor activity in said cell(s).

### BACKGROUND TO THE INVENTION

The main focus in the medical consideration of immune responses has typically been on the responses to pathogens or parasites. Strategies for improving patient outcomes are typically directed at producing or enhancing responses against such entities. In contrast, the present invention is more closely connected with the area of ‘undesirable’ responses. Examples of phenomena where undesirable responses are important include in organ transplantation, autoimmune diseases, recurrent abortion and other conditions which are based upon an underlying inappropriate or illegitimate immune response.

The conversion of pro-inflammatory T cells into cells with regulatory phenotype may be susceptible of exploitation for therapeutic use. In principle, such an approach should allow strategies to halt undesirable immune responses to be developed. However, the progress in this area has been surprisingly slow¹⁰. Even despite the fact that it was demonstrated relatively early on that T_{H} cells ectopically constitutively expressing Foxp3 (T_{H}::Foxp3) can be used to suppress the development of colitis in lymphopenic hosts³, progress has been difficult. It was noted that the effectiveness of polyclonal T_{H}::Foxp3 cells in this context might have been due to the regulation of homeostatic expansion of the co-transferred pro-inflammatory cells, rather than to a true antigen-specific suppression¹¹.

To date, all successful attempts to use T_{H}::Foxp3 in a therapeutic fashion have been limited to the conversion of TCR transgenic T_{H} cells^{7,8}, or experimentally expanded, antigen experienced, clonal populations of T_{H} cells⁹. These approaches ensured that the specificity of the T_{H}::Foxp3 cells matched the specificity of the immune response which was to be suppressed.

Regulatory T cells suppress undesirable immune responses. Under normal circumstances they prevent both autoimmunity¹ and the rejection of the fetus by the maternal immune system². Their development is regulated by Foxp3, a member of the forkhead box family of transcription factors^{3,4,5,6}. Ectopic expression of Foxp3 in pro-inflammatory CD4⁺Foxp3⁻ T cells confers regulatory T cell phenotype, opening a new avenue for therapeutic intervention to prevent autoimmune responses and transplant rejection. However, progress in this area has been surprisingly slow mostly relying on T cell receptor transgenic systems^{7,8} or antigen expanded clonal T cell populations⁹ to demonstrate a beneficial effect.

The invention seeks to overcome problem(s) associated with the prior art.

### SUMMARY OF THE INVENTION

As noted above, it is known that constitutive expression of Foxp3 in a T-helper cell is both necessary and sufficient to convert that cell to a regulatory T cell phenotype. Indeed, there are some techniques available in the prior art which allow a degree of induction of endogenous Foxp3. However, these approaches have problems associated with them such as generating cells which are CD62L low and so therefore display incorrect homing behaviour. In addition, such techniques are typically based on a sub-optimal activation approach and can lead to an unstable induction of Foxp3. Once those cells are reintroduced into the subject, Foxp3 may be turned off again, with no way of turning it back on in vivo.

By contrast, the present inventors have created systems for induction of lineage factors such as Foxp3. In other words, cells can be prepared in such a manner that a lineage factor may be switched on or off within those cells as desired by the operator. It has been discovered by the inventors that such inducible lineage factors have surprising technical effects which would not have been expected from an understanding of the prior art use of lineage factors in various constitutive expression systems. One such unexpected effect is that when the lineage factor is iFoxp3, and its induction is used to convert a T-helper cell to a regulatory T cell, that the homing behaviour of the cells prior to induction is not affected.

Effects such as these allow astonishing medical benefits to be generated. For example, by preparing a cohort of T-helper cells which are capable of being converted into regulatory T cells, the natural homing behaviour of those T-helper cells can be exploited. The T-helper cells are reintroduced into the subject, and are allowed to home to the secondary lymphoid organs and to the site of an inappropriate immune response which it is desired to inhibit. For example, T-helper cells typically migrate to the sites of inflammation in arthritis and the draining lymphoid organs. Then, by administration of the inducing agent, those cells which actively participate in the response are converted into regulatory T cells. The regulatory T cells are thus at the sites where the undesirable immune response is initiated/maintained/acting. These and other benefits flow from the inducible cell switching aspects of the present invention.

The invention is based upon these surprising findings.

Thus in one aspect the invention provides a method of switching the phenotype of a target cell, said method comprising inducing lineage factor activity in said cell via a transgene.

The phenotype of the target cell may comprise the lineage commitment i.e. the differentiation or developmental fate of the target cell.

In another aspect, the invention relates to a method of switching the phenotype of a target cell, said method comprising

(i) introducing to said cell a genetic element capable of inducibly generating lineage factor activity, and
(ii) inducing lineage factor activity in said cell.

Suitably the target cell is a T cell.

Inducibility of the lineage factor activity (as opposed to constitutive activity) is a key feature of the invention.

It is a key feature that the introduction of the transgene and the induction of the lineage factor activity are distinct, separate or discrete events. If the transgene constitutively produces lineage factor activity then this would be inappropriate since it would involve the problems associated with prior art constitutive expression of lineage factor activity. The constitutive expression of lineage factor polypeptide itself is consistent with the present invention, provided that the activity of polypeptide so expressed is inducible.

Suitably said transgene comprises a nucleotide sequence encoding a polypeptide having lineage factor activity. In this embodiment induction of activity may simply be induction of expression of the active polypeptide.

Suitably said transgene comprises an inducible lineage factor. In these embodiments, the lineage factor polypeptide may or may not be constitutively expressed—what is important is that the activity of the lineage factor itself is inducible e.g. by bringing about a change in conformation, post-translational modification, subcellular localisation or other such property of the lineage factor to elicit its activity. This means that the lineage factor itself may persist in an inactive state and that the activity thereof may be induced separately from its expression/presence.

Suitably said transgene encodes a lineage factor fused to a polypeptide capable of controlling the sub-cellular localisation of said lineage factor. Suitably said control polypeptide is an oestrogen receptor polypeptide. Suitably the oestrogen receptor polypeptide is an ERT polypeptide as described below. Suitably such an oestrogen receptor is a modified oestrogen receptor such as a modified oestrogen receptor which does not respond to oestrogen, but rather responds to another compound such as tamoxifen, having the advantage of ameliorating unpredictability due to hormone fluctuations. Most suitably such an oestrogen receptor is a modified receptor which responds only to tamoxifen. Most suitably such an oestrogen receptor has the sequence of one of the oestrogen receptor sequences comprised by a sequence in the sequence listing. Other induction systems may be used if desired.

Suitably said lineage factor is a DNA-binding factor.

Suitably said lineage factor is a transcription factor.

Suitably said lineage factor is Foxp3.

Suitably said target cell is a T cell.

Suitably said T cell is a CD4+ T cell.

Suitably said T cell is a CD8+ T cell.

Suitably said phenotype is switched to a regulatory T cell phenotype following induction of lineage factor activity. In particular this may be brought about when the lineage factor is Foxp3.

In another aspect, the invention relates to a nucleic acid comprising a nucleotide sequence encoding a lineage factor fused to a nucleotide sequence encoding a polypeptide capable of controlling sub-cellular localisation.

In another aspect, the invention relates to a nucleic acid as described above, wherein said lineage factor is Foxp3.

Suitably said nucleic acid comprises Foxp3 and an oestrogen receptor sequence such as the ERT sequence. Suitably said nucleic acid comprises the sequence encoding the Foxp3-ERT fusion comprised by SEQ ID NO:3. Suitably said nucleic acid comprises SEQ ID NO:3.

In another aspect, the invention relates to a nucleic acid as described above, wherein said control polypeptide is an oestrogen receptor polypeptide.

In another aspect, the invention relates to a nucleic acid as described above, wherein said lineage factor is further fused to a nucleotide sequence encoding a fluorescent protein.

In another aspect, the invention relates to a cell comprising a nucleic acid as described above.

In another aspect, the invention relates to a method of suppressing an immune response in a subject, said method comprising inducing lineage factor activity in a target cell of said subject. Said target cell may be in the subject at the time of induction or induction may be conducted ex vivo. Suitably said cell is in the subject at the time of induction.

In another aspect, the invention relates to a method of treating an immune disorder in a subject, said method comprising suppressing an immune response as described above. Suitably said disorder is selected from the group consisting of autoimmune disease, lupus, arthritis, vasculitis, graft vs host disease, transplant rejection, chronic infection, hypersensitivity reaction, asthma, allergies, and recurrent abortion syndrome. Clearly the particular configuration of the treatment should be determined by the operator with consideration of the subject being treated. For example, due to the contraceptive effects of tamoxifen, a tamoxifen inducible system is preferably not used in the context of recurrent abortion syndrome—an alternative induction system is thus preferably selected in such a context.

In another aspect, the invention relates to a cell comprising an inducible lineage factor transgene. Suitably the inducible lineage factor transgene encodes a lineage factor polypeptide which is itself inducible to provide lineage factor activity e.g. by induction of the polypeptide from an inactive to an active state.

Suitably the nucleic acids described above comprise iFoxp3 as shown in SEQ ID NO:3. Suitably the inducible lineage factor comprises the iFoxp3 polypeptide encoded within SEQ ID NO:3.

### DETAILED DESCRIPTION OF THE INVENTION

### Definitions

Abbreviations used may include 4-OHT=4-hydroxytamoxifen; cII=Chicken Collagen TypeII; CIA=Collagen Induced Arthritis; EAE=Experimental Autoimmune Encephalomyelitis; ERT2=mutated estrogen receptor sensitive to tamoxifen but not estrogen; Foxp3=Forkhead box p3; GCV=Ganciclovir; iFoxp3=inducible Foxp3; IRES=Internal Ribosomal Entry Site; MFI=Mean Fluorescence Intensity; MLV=Murine Leukemia Virus; Ova=Ovalbumin; Tam=Tamoxifen; T_{H}=Helper T cell; T_{H}::iFoxp3=CD4+CD25− T cell transduced with iFoxp3; T_{H}::Foxp3=CD4+CD25− T cell transduced with Foxp3; T_{H}::control=CD4+CD25− T cell transduced with a control gene; T_{R}=Regulatory T cell.

The expression ‘illegitimate immune responses’ refers to immune responses which should not occur as they are directed against self.

The expression ‘undesirable immune responses’ refers to immune responses which are directed against legitimate targets (e.g. foetus, transplants) or illegitimate targets (e.g. autoantigens) and have undesirable effects for the host.

Desirable, but illegitimate, immune responses are considered to be immune responses which are directed against illegitimate targets (i.e. selfantigens), but which would have a desirable effect (e.g. attacking cancer cells).

A ‘lineage factor’ is a factor such as a DNA binding factor which alters the lineage commitment of a cell type. (Lineage factors may occasionally be referred to as lineage markers or lineage switches.)

‘Cell type switching’ refers to altering or inducing the lineage commitment of a particular cell type into another cell type (e.g. T_{HO} to T_{Reg}, or T_{H1} to T_{Reg}, or T_{H17} to T_{Reg}, or T_{Reg} to T_{H1}, or T_{HO} to T_{H17}, etc.). This may be accomplished by induction and/or conversion.

If an inappropriate or illegitimate immune response is causing a pathology in the subject, one possible approach might be to supply regulatory T cells. However, the simple ex vivo preparation of regulatory T cells and supply of those T cells to the subject involves numerous problems. Firstly, there are problems of specificity. For example, there can be no guarantee that a mixed population of a regulatory T cell (T-regs) would possess enough, or indeed any, having a correct specificity. Furthermore, dealing with the issue of timing would present serious problems. When should the T-regs be administered? When should the T-regs be prepared? In addition to these problems, there is the issue of location of the cells. T-regs prepared ex vivo typically lose/change their homing abilities. Furthermore, they are typically CD62L low, and as a consequence of this are likely to end up in the liver of the subject rather than at the site of inflammation or inappropriate immune response. Thus, the simple supply of T-regs is insufficient to address these problems. By contrast, a solution provided by the present invention is the provision of inducible cells which can be induced to switch lineage at the desire of the operator. Specifically, one example of the application of the invention is the provision of T-helper cells which can be switched to T-regs by induction of lineage factor(s) in said cells. In this way, the natural multiplication and homing abilities of the T-helper cells is preserved and exploited to populate the area of inflammation or inappropriate immune response with T-helper cells. Then, following induction of switching in those cells, an expanded and localised population of T-regs is created, which population is already expanded and located at the site of the immune response which is desired to inhibit. Such advantageous effects are not possible with prior art approaches.

It is a key feature of some aspects of the invention that T-helper cells are able to take part in the immune response before lineage switching is induced. If T-regs were manufactured and introduced to the subject as T-regs, those would need to be antigen specific, and to be expanded, and then to be introduced into the patient. However, this is a very labour intensive procedure. Furthermore, it is not a beneficial approach. T-regs produced and introduced into a subject in this manner are not at the site of the response. Furthermore, when those cells are reintroduced to the subject, they are CD62L low and therefore exhibit inappropriate homing behaviour.

By contrast, the present invention offers a controlled technique for suppression or control of inappropriate immune responses. Primarily, this control is effected by the administration or withdrawal of the inducer. When the Foxp3-ERT fusion is the inducible lineage factor of the invention, then the inducer is typically tamoxifen.

### Selectable Markers

Suitably the invention may advantageously include the incorporation of one or more selectable markers in combination with the lineage factor of the invention. This has the benefit of permitting selection of those cells into which the inducible lineage factor(s) have been introduced. In particular, selectable markers could be florescent proteins (e.g. GFP), non-immunogenic surface markers (e.g. Thyl), enzymatic markers (e.g. luciferase) or metabolic selection genes (e.g. HisD).

Selectable markers may also be capable of killing or preserving the cell under appropriate selective/inductive conditions—so-called ‘suicide genes’.

Suitably, the invention may advantageously include the incorporation of one or more suicide genes in combination with the inducible lineage factor of the invention. This has the advantage that the cells bearing the inducible lineage factor may conveniently be removed from the patient by activation of the suicide gene should that be deemed advantageous. In this embodiment, removal is by means of a dissection of the cells. One benefit of this approach is that if any of those altered cells became dysregulated and/or cancerous, then each of those cells could be conveniently removed from the patient simply by activating the suicide gene or genes incorporated therein. Suitably, the suicide gene may be the Herpes Simplex thymidine kinase gene (TK gene). In this embodiment, suitably administration of gancyclovir (e.g. Zovirax™) may be used to remove the cells of the invention since those cells expressing the TK gene are killed by the presence of gancyclovir.

The inclusion of a suicide gene is also advantageous in enabling the selective removal of the target cells such as the switched cells. Removal in this context means disabling or killing the cells such as via the suicide gene/selective agent. The cells need not be physically removed so long as they are functionally removed. One advantage of being able to selectively remove the target cells is to alleviate the need for continuous induction treatment. If induction is withdrawn, the cells might revert back to their pre-switched state (e.g. TH::iFoxp3 cells might revert to T effector cells), which may be undesirable or even detrimental. Thus, advantageously one or more selectable marker(s) such as suicide gene(s) are incorporated with the inducible lineage factor(s) of the invention.

Any suitable suicide gene known to those skilled in the art may be employed. Suitably the thymidine kinase (‘TK’) gene is used. In this embodiment, suitably gangcyclovir is used as the selective agent.

Suitably, the suicide gene and the inducible lineage factor are introduced to the cell at the same time e.g. simultaneously. This has the advantage of ensuring that the target cells receive both elements.

Suitably, the inducible lineage factor and the suicide gene may be carried on the same genetic construct. In this embodiment, the safety profile is still further improved since by retaining the inducible lineage factor and the suicide gene on the same genetic construct, any genetic or cell division events which might lead to the separation of the suicide gene from the inducible lineage factor are advantageously minimised.

Suitably, only cells harbouring the suicide gene are administered to a subject. Selection of such cells may be performed if desired, for example by any genetic selection means known to those skilled in the art. This may advantageously include provision of a selectable marker gene on the genetic construct harbouring the suicide gene. Selection may be visual e.g. using a fluorescent protein marker or enzymatic marker.

### Induction

Induction of the cell switching by induction of the inducible lineage factor may be accomplished by any suitable means known to those skilled in the art. This may be by modulation of expression of the lineage factor, or may be by modulation of the location or state of the lineage factor where it is already expressed. For example, when the inducible lineage factor is a Foxp3-ERT fusion, then suitably that protein is constitutively expressed in the cells to be switched. However, in the absence of tamoxifen, the protein would be confined to the cytoplasm. Since Foxp3 is a DNA-binding factor, it is only fully active when present in the nucleus. Thus, administration of the inducer tamoxifen results in translocation of the Foxp3-ERT protein from the cytoplasm to the nucleus, and thus activation leading to cell switching to a T-reg phenotype.

We show that the invention can be applied to techniques such as adoptive transfer of naïve, poly-clonal, wild type T cells transduced with a retroviral transgene carrying an inducible Foxp3 (iFoxp3) (e.g. tamoxifen-inducible Foxp3), and thus enable suppression of immune responses' at will. In contrast to constitutively active wild type Foxp3, iFoxp3 does not alter the homing behaviour of the cells, thus allowing them to participate in immune responses in the same way as they would in the absence of Foxp3. Crucially, it is the inducibility of the system which provides excellent technical benefits, particularly in contrast to prior art systems which are based on constitutive expression and therefore are not inducible. By way of illustration, when Foxp3 is the lineage factor, only once iFoxp3 is induced do the cells assume regulatory T cell phenotype and start to suppress the response they partake in.

Induction may suitably be controlled by any suitable means known to those skilled in the art. For example, induction may be controlled by one or more techniques set out in Weber and Fusenegger (2004 Curr. Opin. Biotech. vol. 15 pp 383-391).

In this or other systems, it may be suitable simply to control the expression of the inducible lineage factor. This may be accomplished by any suitable expression system known in the art. For example, the RheoSwitch® mammalian inducible expression, system (New England Biolabs Inc.) may be used, or one or more transcriptional regulation systems available from Quadrant Biosystems (Intrexon Corporation) may be used.

As an extra safety measure, or in order to provide an especially tight regulation, multiple levels of induction may be built into the system. For example, a Foxp3-ERT fusion might be placed under the control of an, inducible promoter. Thus, the possibility of accidental induction is drastically reduced since two induction events would need to take place, namely induction of expression of the fusion protein, followed by an administration of tamoxifen to facilitate translocation of the expressed protein from the cytoplasm to the nucleus.

Of course, for reasons of simplicity and economy, it may be desirable to have only one level of control of the induction of the inducible lineage factor. It is envisaged that for the great majority of applications, a single level of control of induction would be adequate.

In principle, any hormone receptor system which works by changing localization into the nucleus would be particularly suitable for this type of induction according to the present invention. Of particular interest will be plant and insect hormones, which are likely to (i) have no side effects on the mammalian hormone system and (ii) are unlikely to be immunogenic.

A particularly suitable inducible system is the fusion of the lineage factor to ERT and addition of tamoxifen to induce. This is an example of induction by control of subcellular localisation.

A similar system using a mutated progesterone receptor with the synthetic steroid RU486 has been developed and may be employed in the present invention, for example as described in Kellendonk C, Tronche F, Casanova E, Anlag K, Opherk C, Schutz G: Inducible site-specific recombination in the brain. Mol Biol 285:175-182, 1999. This publication is hereby incorporated herein by, reference, specifically with reference to the sequence and construction of the mutated progesterone receptor, and the nature and dosing of the RU486 inducer.

The RheoSwitch™ inducible system, which relies on a synthetic hormone system, for example as supplied by New England Biolabs Inc. (e.g. Cat. No. E3000S) may also be used in the invention.

Induction may be systemic. In this embodiment, typically the inducer would be administered to the subject as a whole. For example, when the tamoxifen is the inducer, then this could be administered orally or by injection into the bloodstream of the subject. This would then result in distribution of tamoxifen throughout the tissues of the subject, and thus would result in a systemic induction.

In another embodiment, localised induction may be employed. For example, the inducer may be localised by means of a patch or by topical administration through a particular site or tissue of the subject. Alternatively, the inducer may be localised by implantation. Implantation may consist of a slow release reservoir, or any other suitable means of controlling the localised release of the inducer. One such embodiment may involve implantation of a small pump to release the inducer locally into an organ such as the liver.

Localised induction can offer advantages over systemic induction. For example, in the case of a liver transplant patient, a systemic treatment might render them susceptible to infection, particularly if their treatment has involved general suppression of their immune system. By advantageously localising the inducer to the liver; for example by implantation of a pump system, then drawbacks of a systemic approach can be avoided.

It should be noted that any cells migrating or being physically removed from a localised site of induction (for example, removal via the bloodstream) would also be taken away from the site of the inducer. Thus, in the absence of the inducer there will be no more induction of the lineage factor, and the cells should revert to their original type, thereby advantageously minimising any inappropriate suppression effects.

When the lineage factor is fused to an oestrogen receptor polypeptide such as the tamoxifen-sensitive ERT sequence, suitably the induction is via administration of tamoxifen. Dosage of tamoxifen will typically be determined by the operator with reference to the guidance given herein. As is well known, dosage may vary depending upon factors such as method of administration and species of subject. Suitably for mammalian subjects such as humans, a typical dose is approximately 0.01 mg/kg, given orally daily.

### Lineage Factor

The term “lineage factor” as used herein has its natural meaning the art. A lineage factor is an entity which exerts an effect on the fate or lineage of a particular cell. In the context of the present invention, lineage factors are suitably factors involved in governing the fate of a T₀ or naïve T cell. A naïve T cell may differentiate along one of a number of lineages. For example, a naïve T-helper cell (sometimes called a T₀ cell) may become a T_{H}1 cell, a T_{H}2 cell, a T_{H}17 cell, or any other type of T_{H} cell.

Suitably the lineage factor may be selected from GATA3, T-bet, Eomesodermin, RORγt (sometimes referred to as ROR_{gamma-t} or ROR_{gt}) and Foxp3. Suitably said lineage factor is inducible.

The lineage factor may be Blimp-1 (Turner et al 1994 Cell vol 77 pp 297-306). Suitably, when it is desired to switch a target cell into an immunoglobulin secreting cell, the lineage factor is Blimp-1.

Suitably, when it is desired to switch a target cell to T_{H}1, the lineage factor is T-bet.

Suitably, when it is desired to switch a target cell to T_{H}2, the lineage factor is GATA3.

Suitably, when it is desired to switch a target cell to T_{H}17, the lineage factor is ROR γ-T.

Suitably, when it is desired to switch a target cell to Treg, the lineage factor is Foxp3.

Suitably, when it is desired to switch a target cell to a cytotoxic T cell, the lineage factor is eomesodermin.

When the target cell is CD8+, suitably the lineage factor is eomesodermin.

When the target cell is CD4+, suitably the lineage factor is selected from the group consisting of GATA3, T-bet, RORγt and Foxp3.

Although the invention relates to lineage factors generally, numerous embodiments of the invention are illustrated with Foxp3 as the exemplary lineage factor. Most suitably, the lineage factor is Foxp3.

Of course, it may be desired to switch a T-helper cell to a regulatory T cell (Treg). In this embodiment, preferably the lineage factor is Foxp3.

Suitably, the lineage factor is chosen with respect to the target cells in which switching will be induced. In this regard, it is clearly important that the lineage factor chosen is active and is able to exert its effects in the target cells. Thus, suitably the use of cognate lineage factors is preferred. By cognate lineage factor is meant that the lineage factor should be from a similar source to the target cells. Suitably, mammalian lineage factors are used in order to bring about switching in mammalian target cells. More suitably, the lineage factor will be from the same mammalian group as the target cells to be switched. Suitably, primate lineage factors are used in order to switch primate cells. More suitably, the lineage factor used is from the same species as the target cells to be switched. Suitably, human lineage factors are used in order to switch human cells. More suitably, the lineage factor may be from the actual subject from which the target cells are also taken. Thus, suitably the lineage factor will be derived from the genetic complement of the actual subject whose target cells will be switched.

Notwithstanding the above, it should be clear to the skilled operator that any lineage factor which is in fact active in the target cells to be switched would be suitable for use according to the present invention. Activity in the target cells may be conveniently and easily tested by attempting switching as described herein. Truncated, modified, chimeric or otherwise altered lineage factors may also be used in the present invention. In case any guidance is needed in identifying lineage factors, reference is made to the exemplary sequences of lineage factors disclosed herein such as in the sequence listing. In this regard, it should be noted that exemplary sequences of RORgt are found in several occurrences in the sequence listing. SEQ ID NO:6 contains a few extra residues which may be discarded; SEQ ID NO:8 contains a preferred RORgt sequence; SEQ ID NO:9 contains a preferred RORgt sequence in a preferred core vector; thus SEQ ID NO:9 also discloses a preferred core vector sequence (i.e. by removing the sequence of SEQ ID NO:8 from the sequence of SEQ ID NO:9 the core vector sequence is obtained). Of course sequence substitutions may be made such as conservative substitutions, or splice variants or alternate alleles may be used provided the key character of the lineage factor is not altered. The key character or key feature which needs to be retained by lineage factor for a particular application is the ability to induce switching in the target cells. As noted above this may be easily tested by attempting switching by induction of the chosen lineage factor in the chosen target cells, and observing those cells to determine whether or not their phenotype is switched. Clearly, a lineage factor which is unable to produce the switching phenotype will be of limited or no use in the present invention. For these reasons, it is important that the lineage factors or fragments thereof which are used in the methods of the invention retain the ability to bring about switching in the target cells.

For example, in relation to Foxp3, there are certain regions that are suitably conserved in order to maintain lineage factor function; thus, other elements of Foxp3 are particularly susceptible to being altered, such as truncated or substituted, provided that the resulting Foxp3 construct retains its function in bringing about switching in the target cells. The particular regions of Foxp3 which should be conserved include: the N-terminal stretch of 150 aa and the C-terminal fork head domain. It is believed that these are very important to the function of Foxp3. It may be of help to note that within the forkhead domain there is a nuclear localization sequence which is believed to be important for the function of the wild type Foxp3, but in the context of the invention the function is modulated through rendering the polypeptide inducible (e.g. via the ERT fusion/application of tamoxifen) so that the naturally occurring nuclear localisation sequence may also be truncated and/or substituted provided its function is retained.

More specifically, in relation to Foxp3 the following guidance is provided as to regions of Foxp3 which should suitably not be substituted or truncated or otherwise altered:

aa 70-151; preservation of this sequence is preferred due to interaction with cREL and possibly other transcription factors.

aa 337-410, the forkhead domain; preservation of this sequence is preferred for NFAT interaction and DNA binding.

aa397; preservation of this residue is preferred for proper function of the forkhead domain.

aa371; preservation of this residue is preferred for proper function of the forkhead domain.

Thus, suitably when the lineage factor of the invention is Foxp3, suitably at least amino acid residues corresponding to aa 70-151, aa 337-410, aa397 and aa371 of wild type Foxp3 are conserved.

Similar analyses may be conducted if it is desired to truncate or vary the sequence of any other lineage factor(s) of the invention.

In some embodiments it may be desired to alter only particular element(s) of a target cell's phenotype. For example, mutant lineage factors may be used to obtain partial effects or one or more subsets of effects relative to the wild-type lineage factor(s). One example of this may be to use an inducible mutant Foxp3 in order to induce the horning behaviour of a Treg yet without inducing the suppressive activity.

As used herein, the term induction as applied to induction of a lineage factor or induction of switching means induction of the lineage factor's activity. In some embodiments, this may be as simple as inducing expression of the lineage factor. If the lineage factor so expressed is indeed active, then mere induction of its expression would be sufficient to induce it, and therefore to induce its activity and thus induce the switching. However, a more sophisticated induction mechanism may be used if desired. For example, some lineage factors may only be active when translocated to a particular sub-cellular compartment. In this situation, the operator may choose to have the lineage factor constitutively expressed in the target cells, and may use an alternative induction mechanism to bring about its activity. One example of this is when the lineage factor is a transcription factor. Transcription factors need to reach the nucleus in order exert their activity. By modifying the lineage factor, for example by fusion to a protein capable of controlling of its sub-cellular localisation or translocation pattern, then in those embodiments induction of activity would correspond to induction of translocation of said modified lineage factor.

It is further possible that the lineage factor may be multi-factorial. In this embodiment, a subset of the elements making up the lineage factor might be constitutively expressed, with modulation of the overall lineage factor activity dependent on induction of expression or induction of translocation of the one missing element required for activity.

It is important to appreciate that whatever the system chosen by the operator for induction of lineage factor activity, it is the activity of the lineage factor which is crucial, rather than a mere presence or absence of said lineage factor. Of course, clearly there are embodiments where the activity of the lineage factor is entirely dependent on its presence or absence. Clearly, one of the simplest ways to induce activity of a lineage factor is simply to induce its expression. Equally clearly, there are embodiments where the lineage factor may be present in the target cells regardless whether or not they have been induced to switch their phenotype, with the induction being designed to alter the behaviour, location, post-translational modification, or other characteristics of said lineage factor in order to modulate its activity.

In all embodiments, it is important to note that it is the activity of the lineage factor which is being manipulated, whether or not that correlates with its mere expression may vary from embodiment to embodiment.

It should be noted that it may be desirable to arrange the lineage factors to permit multiple switching events. In a first embodiment, this may be accomplished simply un-inducing or switching off the activity of the lineage factor. This typically leads to reversion of the switched cell to its original state. In another embodiment, it may be desirable to switch the cell a second time, to turn it into a third cell type. For example, a T₀ cell may be initially switched to a Tₕ1 cell by activation of an appropriate lineage factor such as T-bet. It may then be desired to switch this Tₕ1 cell to a regulatory T cell, for example by induction of Foxp3 activity. These and other combinations featuring the various factors and switching methods described herein are intended to be within the scope of the present invention.

### Target Cells

The target cell may be any immune cell for which it is desired to switch type. Suitably the target cell is a cell of the T-cell lineage, i.e. suitably a T-cell. Suitably said cell is a naïve T cell (sometimes referred to as a T₀ cell).

Naïve T cells are cells which have been produced (and have survived the positive and negative selection in the thymus) but is regarded as not yet having encountered antigen. Naïve T cells are considered to be mature but are not yet activated/expanded due to not yet having encountered cognate antigen. Suitably the target cell of the invention is a naïve T cell. Naïve T cells are typically characterised by expression of CD62L (sometimes referred to as L-selectin), and/or the absence of activation markers such as CD25, CD44 or CD69. An advantage of the target cells being naïve T cells is that they are not yet activated or committed to a particular path of differentiation and can be switched from the Tₒ or ‘ground’ state.

Suitably the target cells comprise a population of polyclonal T cells. Suitably said cells are as harvested from peripheral blood.

As explained above, the target cells may be T cells which have already proceeded along a particular lineage. For example, the target cells may be T cells which have already developed into T helper (T_{H}) cells, or into regulatory T cells (Tregs). Within these classes, the target cells may be further committed e.g. they may have assumed a particular T_{B} type such as T_{H}1, T_{H}2, T_{H}17 or other type. It is an advantage of the invention that these cells may also be target cells and may be switched according to the present invention. This is particularly advantageous for embodiments taking advantage of the characteristics of particular T_{H} cell phenotypes for example the homing behaviour of T_{H} cells before switching to a non-T_{H} cell type takes place. Furthermore, this advantageously provides an even greater flexibility in application of the invention.

For example, if it is desired to produce a T_{H}2 cell, this may be accomplished according to the present invention in an number of ways. Firstly, a T₀ cell may be switched to a T_{H}2 cell, e.g. by inducing lineage factor such as GATA-3. Secondly, a T_{H}17 cell may be switched to a T_{H}2 cell, for example by inducing lineage factor such as GATA-3. Alternatively, if the T_{H}2 cell had previously been switched to another cell type such as Treg by induction of a lineage factor such as Foxp3, then induction may be withdrawn, allowing the cell to revert and thereby creating (e.g. recreating/reverting) a T_{H}2 cell in that manner. Thus, it can be appreciated that the invention may be advantageously applied in a number of different ways, the key underlying technical connection being the switching of cell type by induction of lineage factor.

The target cell may be a CD4+ cell, a CD8+ cell or a naïve cell from the bloodstream. Thus, the target cell may be a T_{H}1, T_{H}2, T_{H}17 or other type of T_{H} cell, a T₀ cell (naïve T cell), a Treg, or a population of cells comprising one or more such cell types e.g. polyclonal T cells such as polyclonal T cells harvested from peripheral blood.

### Transfection/Transduction

Delivery of the nucleic acids of the invention to cell(s) is suitably accomplished using a vector. Such vectors are well known in the art. Any vector permitting introduction of the nucleic acid of interest into a cell may be employed. Suitably viral vectors are used. Suitably retroviral or DNA based viral vectors may be used. Most suitably the viral vector is or is derived from a lentivirus based vector.

In the examples section m6p based vectors are described. These vectors are based on the Moloney Murine Leukemia Virus (MLV)—a retrovirus which is capable of infecting dividing cells. m6p vectors are vectors in which all the structural genes have been taken out, and only the ‘Long Terminal Repeats’ (LTRs) remain. The requisite structural genes are provided in trans. An overview of the different viruses can be seen in FIG. 4. Furthermore such viral vectors contain an ‘Internal Ribosomal Entry Site’ (IRES) to drive the protein expression of markers (such as GFP).

“Cell transfection” refers to the introduction of foreign or exogenous nucleic acid into a cell. There are several methods of introducing DNA or RNA into a cell, including chemical transfection methods (e.g. liposome-mediated, non-liposomal lipids, dendrimers), physical delivery methods (e.g. electroporation, microinjection, heat shock), and viral-based gene transfer such as viral transduction (e.g. retrovirus, adeno-associated virus, and lentivirus). The method of choice will usually depend on the cell type and cloning application and alternative methods are well known to those skilled in the art. Such methods are described in many standard laboratory manuals such as Davis et al, Basic Methods In Molecular Biology (1986).

Transfected genetic material can either be,expressed (whether constitutively or inducibly) in the cell transiently or permanently. In transient transfection, DNA is transferred and present in the cell, but nucleic acids do not integrate into the host cell chromosomes. Typically transient transfection results in high expression levels of introduced RNA 24-72 hours post-transfection, and DNA 48-96 hours post-transfection. Stable transfection is achieved by integration of DNA vector into chromosomal DNA and thereby permanently retaining said nucleic acid in the genome of the cell.

Chemical means of transfecting cells with foreign nucleic acid include use of DEAE-dextran, calcium phosphate or artificial liposomes. DEAE-dextran is a cationic polymer that associates with negatively charged nucleic acids. An excess of positive charge, contributed by the polymer in the DNA/polymer complex allows the complex to come into closer association with the negatively charged cell membrane. It is thought that subsequent uptake of the complex by the cell is by endocytosis. This method is successful for delivery of nucleic acids into cells for transient expression. Other synthetic cationic polymers may be used for the transfer of nucleic acid into cells including polybrene, polyethyleneimine and dendrimers.

Transfection using a calcium phosphate co-precipitation method can be used for transient or stable transfection of a variety of cell types. This method involves mixing the nucleic acid to be transfected with calcium chloride, adding this in a controlled manner to a buffered saline/phosphate solution and allowing the mixture to incubate at room temperature. This step generates a precipitate that is dispersed onto the cultured cells. The precipitate including nucleic acid is taken up by the cells via endocytosis or phagocytosis.

Transfection using artificial liposomes may be used to obtain transient or longer term expression of foreign nucleic acid in a host cell. This method may also be of use to transfect certain cell types that are intransigent to calcium phosphate or DEAE-dextran.

Liposomes are small membrane-bound bodies that can actually fuse with the cell membrane, releasing nucleic acid into the cell. A lipid with overall net positive charge at physiological pH is the most common synthetic lipid component of liposomes developed for transfection methods using artificial liposomes. Often the cationic lipid is mixed with a neutral lipid such as L-dioleoylphosphatidyl-ethanoloamine (DOPE). The cationic portion of the lipid molecule associates with the negatively charged nucleic acids, resulting in compaction of the nucleic acid in a liposome/nucleic acid complex. Following endocytosis, the complexes appear in the endosomes, and later in the nucleus. Transfection reagents using cationic lipids for the delivery of nucleic acids to mammalian cells are widely available and can be obtained for example from Promega (TransFast™ Transfection Reagent).

In addition to the above, transduction, for example using viral vectors, may suitably be accomplished by retroviral transduction of target cells using vectors based on MMLV (murine) or HIV (primate); this results in permanent incorporation of the gene into target cells. Other viral methods operating in a similar manner include AAV (adeno-associated virus). Adenovirus may also be used, for example to produce transient expression.

### Further Applications

The invention may be applied in the suppression of undesirable immune responses using polyclonal T cells transduced with inducible lineage factor such as Foxp3.

The methods and techniques described herein find application in treatment of non-desirable immune responses such as auto-immune diseases. For example, diseases in which regulatory T cells have the potential to stop the response, but for some reason fail to do so. Clearly, the prevention of transplant rejection is one of the most important applications of the invention.

The advantages of our strategy are many fold. It may advantageously use polyclonal, naïve T cells. It does not require any prior knowledge of the antigen specificities involved, a prior art problem which complicates the ex vivo expansion of regulatory T cells for therapeutic use^{21,22,23}.

Furthermore, our approach does not rely on any endogenous triggers, although of course the existence of an undesirable response (i.e. the pathology being addressed) may in a strict sense be regarded as an endogenous trigger. As we externally trigger the phenotypic conversion of the cells by induction of lineage factor activity, the exact time point when this happens can be determined by the operator.

The invention may be applied to restrict the induction of suppression to a geographically defined region by local administration of the inducing agent.

By use of either or both such temporal and spatial controls advantageously enables prevention or reduction of collateral damage, which might be caused by a more systemic immunosuppression. Of course in other embodiments systemic immunosuppression may be desired.

The safe utilization of gene therapy is an established, and of course evolving, area^{24,25} and thus this approach to transgene delivery to the target cells is well within the abilities of the skilled user.

Thus the invention provides strategies to specifically inhibit undesirable immune responses in subjects such as humans.

The invention may be applied to treatment or prevention of diabetes.

In one embodiment the invention may relate to a method of inducibly lowering the expression of CD62L in a cell, said method comprising inducing lineage factor activity in said cell.

The requirement of the invention to use inducible lineage factor activity provides advantages as set out herein. Furthermore, the ‘disguised’ nature of the cells before switching can be exploited. For example, T_{H} cells harbouring inducible Foxp3 lineage factor activity behave as normal T_{H} cells before induction/switching. Thus they go through normal self selection and expansion upon encountering antigen. This is an advantage because then precisely those cells which will be switched have already expanded ‘naturally’ in the host. Thus there are advantageously more of those cells pre-switching due to natural expansion and selection. Furthermore, switching not only has the advantage of providing Tregs at the site of the response thereby suppressing the response locally as desired, but also has the effect of removing T_{H} cells from the site of the response (due to switching them to Tregs, thereby ‘removing’ each T_{H} cell which is switched—of course the cell is not removed but after switching it is no longer a T_{H} cell so has effectively been ‘removed’ as a T_{H} cell.

Furthermore, the invention finds application from the reversion/reversible nature of the inducible switching. Tumours tend to accumulate Tregs within the tumour itself. This can contribute to immune evasion by suppression of immune responses directed against the tumour. This is clearly undesirable. According to the present invention, cells may be switched to Treg within the patient. These are then allowed to accumulate in the tumour according to the natural process. Once the tumour is populated with switched Tregs, then induction may be withdrawn i.e. the cells may be switched back to T_{H} cells. This has the twin advantage of ‘removing’ suppressive Tregs from the tumour (i.e. removing them by switching them to another type rather than physical removal as explained above), but also creates T_{H} cells within the tumour, thereby provoking or enhancing a helpful immune response against the tumour as well as alleviating suppression of that response by the (pre-switching) Tregs.

In another aspect, the invention relates to a, method of enhancing and/or biasing an immune response in a subject, said method comprising inducing lineage factor activity in a target cell of, said subject. In another aspect, the invention relates to a method of biasing and or boosting an insufficient or inappropriate immune response in a subject, said method comprising enhancing an immune response as described above. Suitably said insufficient immune response is in the context of vaccination, infection (such as viral, bacterial, fungal, or parasitic infection), or cancer. Clearly, although the invention has been illustrated with an array of immune suppressive or immune diverting effects, the invention also finds application in the enhancement of immune responses. For example, it is a benefit of the invention that undesirable or illegitimate immune responses may in fact be enhanced by the use of inducible lineage factors as taught herein. This can be advantageous for example in the augmentation of responses against tumours or other pathological entities which might bear ‘self’ antigens and thus represent a context in which enhancement of an otherwise illegitimate or undesirable immune response is in fact therapeutically useful.

In another aspect the invention may advantageously be combined with Tolerostem™ cells produced using Medistem Laboratories Inc. systems.

The invention may also be used in overriding polarisation signals such as Th1 polarisation signals. For example, when the lineage factor is RORgt, IFNgamma may be suppressed and Th17 may be promoted. This effect is advantageously dominant over external stimuli. This finds application in disease settings where pathogens have evolved to slip the immune system, for example where the pathogen is a bacterium and a Th2 response is needed but the bacterium ‘fools’ the immune system into a Th1 response and thus evades clearance. The invention may advantageously be used in this context to force the response in the correct direction, particularly when the lineage factor is RORgt.

### BRIEF DESCRIPTION OF THE FIGURES

FIG. 1 shows graphs and charts demonstrating that constitutive Foxp3 transduced cells fail to suppress collagen-induced arthritis and exhibit altered homing behaviour. (a, b) Arthritis was induced on day 0 by immunization with chicken collagen in Complete Freund's Adjuvant. (diamonds) Mice did not receive any further treatment; n=18. (circles) Mice received 10⁶ T_{H}::Foxp3 cells one day prior to disease induction; n=7. The progression of the disease was monitored blindly on a daily basis by scoring the inflammation of the paws (0—no swelling, 1—swelling in individual joint, 2—swelling in more than one joint or mild inflammation of the paw; 3—severe swelling of the entire paw and/or ankylosis). The scores for all paws of each mouse were totaled (maximum reachable score of 12 per mouse). Mice reaching a score of 8 or more were euthanized. All the experiments were stopped at day 51. (a) The average arthritis scores of all mice in the groups are shown for each day. (b) Maximum arthritis score reached by each of the animals. (p values were determined using Fischer's Exact test) (c-f) comparison of the homing behaviour of T_{H}::Foxp3, T_{H}::control, T_{H} and T_{R} cells. 10⁶ cells were transferred into each mouse (control, n=4; Foxp3, n=6; T_{H}, n=3; T_{R}, n=3). After 48 h the various tissues were collected and analysed by flow cytometry, the transduced cells were identified based on their expression of GFP, primary cells were CFSE labelled. (c, e) frequency [%] at which the transferred cells can be found in each of tissue. (d,f) Relative distribution of the transferred cells within the various tissues. (g,h) CD4⁺CD25⁻ cells were activated for 36 h and then transduced (0 h) with either a control (black line, n=3) or Foxp3 (red line, n=3). (g) CD62 surface expression at 0 h and 24 h after transduction (h) Percentage of surfaceCD62L^{hi} cells was analysed in the transduced populations at the indicated time points (mean of three independent experiments). All error bars represent standard error of the mean.

FIG. 2 shows scatterplots, charts and graphs of effects of inducible Foxp3 (tamoxifen-induction). (a-d) Comparison of CD25 and CD62L surface expression in cells transduced with either a control gene, Foxp3, or iFoxp3. The transduced cells were identified based on the co-expression of rat CD8 (a, c) Representative FACS profiles determining the (a) level of CD25 or (c) surface CD62L expression of the transduced cell populations. (b),Mean intensity of CD25; n=2 and (d) percentage of surfaceCD62L^{hi} cells amongst the Foxp3 and iFoxp3 transduced cells; n=2. (e) change in surface CD62L expression on TH::iFoxp3 cells at various time points after induction with 50 nM 4-hydroxytamoxifen. (1) Proliferation of control, Foxp3 and iFoxp3 transduced cells measured by ³H thymidine incorporation in absence (white bars, n=3) or presence of 50 nM 4-hydroxytamoxifen (grey bars, n=3). (g) Time course measuring the suppressive activity of T_{H}::iFoxp3 cells upon addition of 50 nM 4-hydroxytamoxifen. 10⁵ CFSE labelled CD4⁺CD25⁻ T cells were incubated with either 10⁵ control transduced T_{H} cells (solid black line (upper line)), or 10⁵ T_{H}::Foxp3 cells (solid red line (lower line)) or 10⁵ T_{H}::iFoxp3 cells (dotted red line (middle line)). In either case, two individual experiments were performed for each time point. The cells were co-cultured from time point 0 h and the proliferation was measured based on CFSE dilution after 72 h. 4-hydroxytamoxifen was added at the various time points indicated. (h) Comparison of the homing behaviour of T_{H}::iFoxp3 (n=3) and T_{H}::control, (n=3). The experiment was performed as outlined in FIG. 1. The relative distribution of the transferred cells within the various tissues is shown. All error bars represent standard error of the mean.

FIG. 3 shows graphs, plots and charts showing that induced T_{H}::iFoxp3 cells suppress collagen-induced arthritis. (a, b) Arthritis was induced and monitored as described in FIG. 1. Mice that did not receive any further treatment (black diamonds), n=18; mice that received 10⁶ T_{H}::iFoxp3 cells one day prior to disease induction (red circles, dotted line), n=7 and mice that received 10⁶ T_{H}::iFoxp3 cells one day prior to disease induction and tamoxifen injections to induce iFoxp3 from day 15 onwards (red circles, solid line), n=25. (a) The average arthritis scores of all mice in the groups are shown for each day. (b) Maximum arthritis score reached by each of the animals. (c, d) T_{H}::iFoxp3 cell can readily be detected in the spleen 52 days after transfer into DBA1 mice, independent of tamoxifen treatment and arthritis level. The cells were identified based on the co-expression of GFP (c) Representative FACS profiles. (d) Summary of the frequency of GFP⁺cells in the spleen 52 days after transfer (n=4 in both cases).

FIG. 4 shows diagrams of retroviral vectors. Foxp3 was amplified from Balb/c cDNA and iFoxp3 was constructed by a C-terminal fusion of ERT2 replacing the Foxp3 stop-codon and cloned into the retroviral vectors m6p_GFP and m6p_rCD8. GFP was fused to the N-terminus of iFoxp3²⁶.293eT cells were co-transfected with pCI-Eco and m6p_GFP or m6p_rCD8 (1:1) carrying a Foxp3, blasticidine-S-deaminase (control), iFoxp3 or GFP-iFoxp3 transgene.

FIG. 5 shows photomicrographs of iFox3p induction in vivo. Sub cellular localization of the GFP-iFoxp3 fusion protein within T_{H}::GFP-iFoxp3 cells which had been injected into mice and sorted four days later by flow cytometry. Mice received each day an i.p. injection of either (a) vehicle or (b) tamoxifen.

FIG. 6 shows graphs demonstrating that tamoxifen treatment has only minor effect on collagen-induced arthritis. Arthritis was induced and monitored as described in FIG. 1. Mice did not receive any further treatment (black diamonds); n=18 mice that received tamoxifen injections from day 15 onwards (triangles); n=14. The average arthritis scores of all mice in the groups are shown for each day.

FIG. 7 shows scatterplots and a bar chart demonstrating tissue distribution of T_{H}::iFoxp3 cells at day 52. Tamoxifen induced T_{H}::iFoxp3 cell can readily be detected in the blood, spleen and auxiliary lymph nodes (aux. LN) at 52 days after transfer into DBA/1 mice (collagen/CFA immunized). The cells were identified based on the co-expression of GFP (a) Representative FACS profiles of tissues. For comparison the representative profiles from mice that had received no cell transfer are shown. (b) Summary of the frequency of GFP⁺cells in various tissues 52 days after transfer (n=4). For comparison blood frequency of GFP⁺cells in blood 17 days after transfer is shown (n=4).

FIG. 8 shows bar charts illustrating the level of arthritis specific IgG antibodies. Comparison of the levels of collagen-specific IgG1, IgG2a, IgG2b and IgG3²⁷ prior to arthritic induction (pre) and at the end of the experiments on day 51 (post) in control mice (white bars) and mice that had received T_{H}::iFoxp3 cells (grey bars). Results are shown as a mean of six randomly chosen animals from each group in.

FIG. 9 shows that T_{H}::iFoxp3 cells partake in the immune response and suppress it upon induction. (A-C) CD4 CD25⁻ T cells were purified from DO11.10xSCID mice and transduced with either Foxp3 or iFoxp3. Balb/c females received i.v. 5×10⁴ transduced and non-transduced cells at a ratio of 2:3 before being immunized with ova in CFA [+ova] or CFA alone [−ova] (n=3 in all cases). Mice were sacrificed eight days after immunization. (A) The frequency of GFP⁺ cells from tissues was measured and the relative expansion was calculated as % GFP⁺ [+ova] % GFP⁺[−ova] for T_{H}::Foxp3 (white bars) and T_{H}::iFoxp3 cells (grey bars).

(B) Total splenocytes were isolated from mice receiving T_{H}::iFoxp3 cells and challenged with the indicated amounts of ova for 72 h in the absence (white bars) or presence (grey bars) of 50 nM 4-hydroxytamoxifen. The total proliferation was measured by the ³H-thymidine incorporation and the relative proliferation was measured as ³H counts/min [+ova]/³H counts/min [−ova]. (C) Total ova-specific antibodies were measured in pre-bleeds (white bars; n=2) and 8 days after immunization (grey bars; n=3) with [+ova] or [−ova].

FIG. 10 shows graphs of average weight per mouse against time.

FIG. 11. Polyclonal T_{H}::Foxp3 cells fail to suppress CIA and exhibit altered homing behavior. (A) Arthritis was induced on day 0 by immunization with ell in CFA. Mice that did not receive any further treatment (black, n=27) and mice that received 1×10⁶ T_{H}::Foxp3 cells one day prior to immunization (red, n=7). The average arthritis scores of all mice in the two groups are shown. (B, C) Comparison of the homing behavior of (B) CFSE labeled T_{H} (black) and T_{R} (red) cells and (C) GFP-expressing T_{H}::control (black) and T_{H}::Foxp3 (red) cells. 1×10⁶ cells were transferred into each mouse (T_{H}, n=3; T_{R}, n=3; control, n=4; Foxp3, n=6) and the tissues were analyzed 48 h later by flow cytometry. The diagrams represent the percentage of cells in each tissue, calculated from the total number of cells recovered in all tissues together (1.2×10⁵±0.1×10⁵ T_{H} cells and 1.1×10⁵±0.2×10⁵ T_{R} cells; 8.3×10⁴±2.7×10⁻⁴ T_{H}:: control cells and 5.1×10⁴±0.9×10⁴ T_{H}::Foxp3: cells; values±SEM). Error-bars represent the SEM.

FIG. 12. Foxp3 mediated regulation of CD62L. (A-D) CD62L expression on CD4⁺Foxp3⁻ T_{H} cells (black) and CD4⁺Foxp3⁺ T_{R} cells (red). (A) Representative FACS profiles for CD62L expression on T_{H} and T_{R} cells prepared from spleen (n=3 in each case) with unstained T_{H} cells (grey) shown as control. (B) Mean fluorescence intensity (MFI) of CD62L on T_{H} and T_{R} cells from indicated tissues (n=2 in each case). (C) Representative FACS profiles of CD4⁺CD25⁻ T_{H} (black) and CD4⁺CD25⁺ T_{R} (red) cells activated for 72 h (n=3 in each case). (D) Total splenocytes were incubated in the absence of any treatment (solid line) or activated by addition of 100 ng/m1 PMA in the presence (dashed line) or absence (dotted line) of 50 μM TAPI-2 (n=3 in each case). (E-I) CD62L expression in T_{H}::control (black) and T_{H}::Foxp3 cells (red). CD4⁺CD25⁻ cells were activated for 36 h and transduced (0 h) with either m6p8[control] (black line; n=3) or m6p8[Foxp3] (red line; n=3). (E, F) Representative FACS profiles of CD62L expression on transduced cells at (E) 0 h and (F) 24 h after transduction. (G) Percentage of CD62L^{hi} cells within the transduced populations in the presence (dashed line) or absence (solid line) of 50 μM TAPI-2. (H) Amount of soluble CD62L in the supernatant measured by ELISA (representative of two independent experiments). (I) Relative CD62L expression in CD4⁺CD25⁻ T_{H} and CD4⁺CD25⁺ T_{R} cells (n=3 in each case), as well as T_{H}::control and T_{H}::Foxp3 cells 48 h after transduction (n=2 in each case) determined by qPCR and normalized to HPRT. Error bars represent the SEM.

FIG. 13. Inducible Foxp3. (A) Diagram of iFoxp3 containing retroviral vectors m6pg[iFoxp3] either co-expressing GFP or a GPI-linked ratCD8 α-chain m6p8[iFoxp3] and m6p8[GFP-iFoxp3] which contains a fusion of GFP and iFoxp3. (B) MFI of intra-cellular stain for Foxp3 in T_{H}::Foxp3 and T_{H}::iFoxp3 cells compared to CD4⁺T_{R} and T_{H} cells (n=2 in each case). (C, D) Sub-cellular localization of GFP-iFoxp3 in T_{H}::GFP-iFoxp3 cells (C) in vitro after 48 h in the presence or absence of 50 nM 4-OHT or (D) in vivo after three injections of tamoxifen or carrier. (E-G) Gain of T_{R} cell function upon induction of iFoxp3. (E) Proliferation of T_{H}::control, T_{H}::Foxp3 and T_{H}::iFoxp3 cells upon antiCD3ε [0.6 μg/ml] stimulation measured by ³H-thymidine incorporation in the absence (white bars; n=3 in each case) or presence of 50 nM 4-OHT (grey bars; n=3 in each case). (F) 1×10⁵ CFSE labeled CD4⁺CD25⁻ target T cells were co-cultured with 1×10⁵ T_{H}::control, T_{H}::Foxp3 or T_{H}::iFoxp3 cells and activated with antiCD3ε [0.6 μg/ml] (n=2 in each case). The proliferation of target cells was measured based on CFSE dilution after 72 h and the % of cells that had undergone at least one cell cycle is shown. The assay was performed in the absence (white bars) or the presence (grey bars) of 50 nM 4-OHT added to the transduced cells 24 h prior to set-up. (G) MFI of CD25 48 h after transduction on T_{H}::control, T_{H}::Foxp3 and T_{H}::iFoxp3 in the absence (white bars; n=2 in each case) or presence of 50 nM 4-OHT (grey bars; n=2 in each case). (H, I) Comparison of CD62L expression on T_{H}::control, T_{H}::Foxp3 and T_{H}::iFoxp3 48 h after transduction with m6p8. (H) Representative FACS profiles of CD62L expression (n=2 in each case). (I) Percentage of CD62L^{hi} cells within the transduced populations. (J) Comparison of the homing behavior of T_{H}::control (black) and T_{H}::iFoxp3 (red) cells. 1×10⁶ cells were transferred into each mouse (T_{H}::control, n=2; T_{H}::iFoxp3, n=3) and the tissues were analyzed 48 h later by flow cytometry. The diagrams represent the percentage of cells in each tissue calculated from the total number of cells recovered in all tissues together (5.4×10⁵±0.7×10⁵ T_{H}::control cells and 3.1×10⁵±0.4×10⁵ T_{H}::iFoxp3 cells; values±SEM).

FIG. 14. T_{H}::iFoxp3 cells partake in the immune response and suppress it upon induction. (A-C) Balb/c mice received 2×10⁴ T_{H}::Foxp3 or T_{H}::iFoxp3 cells prepared from DO11.10xSCID mice before being immunized s.c. with either ova in CFA [+ova] or CFA alone [−ova] (n=3 in each case). (A) The frequency of GFP⁺ cells was measured eight days after immunization and the relative expansion was calculated as % GFP⁺[+ova]/% GFP⁺[−ova]. (B) Total ova-specific antibodies in pre-bleeds (d0, white bars; n=2 in each case) and 8 days after immunization (d8, grey bars; n=3 in each case) in immunized and naïve mice. (C) Total splenocytes were isolated from mice which had received T_{H}::iFoxp3 cells and were challenged with the indicated amounts of ova for 72 h in the absence (white bars) or presence (grey bars) of 50 nM 4-OHT. The total proliferation was measured by ³H-thymidine incorporation and the relative proliferation was calculated as [+ova]/[−ova]. (D, E) Mice received 1×10⁶ polyclonal T_{H}::iFoxp3 cells and were immunized s.c. with ova in CFA. A week later various tissues were analyzed. (D) The total number of recovered T_{H}::iFoxp3 cells from immunized mice (red, n=3) or non-immunized mice (black, n=3) was calculated. (E) The relative number of endogenous and T_{H}::iFoxp3 cells was calculated as a ratio between immunized and non-immunized mice. All error bars represent SEM and p values were determined using an unpaired t test.

FIG. 15. T_{H}::iFoxp3 cells suppress collagen-induced arthritis upon iFoxp3 induction. (A, B) Arthritis was induced on day 0 by immunization with cII in CFA. (A) Mice that received 1×10⁶ T_{H}::iFoxp3 cells (grey, n=17), mice that did not receive any further treatment (black, n=27), mice that received tamoxifen injections (tam) (blue, n=14) and mice that received 1×10⁶ T_{H}::iFoxp3 cells and tamoxifen injections to induce iFoxp3 (red, n=25). The average arthritis scores of all mice in the groups are shown for each day. (B) Maximum arthritis score reached by individual animals, that had received no transfer of cells, T_{H}::Foxp3 cells (see FIG. 11A) and T_{H}::iFoxp3 cells+/−tam. (C, D) Arthritis was induced by immunization with cII in CFA. (C) Mice that had received 1×10⁶ T_{H}::iFoxp3 cells the day before ell immunization and tamoxifen injections (red, n=4) when the mice reached a score of 3 (day 0) and mice that did not receive any further treatment (black, n=9). (D) Maximum arthritis score reached by individual animals. Error bars represent the SEM and p values were determined using Fisher's Exact Test.

FIG. 16. T_{H}::iFoxp3 cell-mediated suppression is specific. (A, B) Mice were immunized with cII in CFA on day 0. (A) On day 35 ex vivo recall reactions to cII were performed on cells purified from mice that did not receive any further treatment (control, n=10), mice that had received 1×10⁶ T_{H}::iFoxp3 cells and tamoxifen injections (T_{H}::iFoxp3+tam, n=10) and naïve mice (naïve, n=10). (B) Some of the mice described in (A) were immunized on day 28 with ova and ex vivo recall reactions to ova were performed in parallel (control, −ova: n=3, +ova: n=7; T_{H}::iFoxp3+tam, −ova: n=3, +ova: n=7; naïve, −ova: n=5, +ova: n=5). (C) Mice were immunized simultaneously with cII and ova in CFA on day 0 and ex vivo antigen-specific recall reactions to ova (closed), ell (half-closed) were performed on day 28. Mice that did not receive any further, treatment (naïve, n=4), mice that received 1×10⁶ T_{H}::iFoxp3 cells and tamoxifen injections (T_{H}::iFoxp3+tam, n=4) and mice that received 1×10⁶ T_{H}::iFoxp3 cells (T_{H}::iFoxp3, n=4). p values were determined using an unpaired t test.

FIG. 17. T_{H}::iFoxp3 cell longevity. (A) Representative FACS profiles of splenocytes purified from the indicated mice 52 days after transfer of 1×10⁶ T_{H}::iFoxp3 cells. (B) Summary of the frequency of GFP⁺ cells in the spleen 52 days after transfer (n=3 in each case). (C) Representative FACS profiles of specified tissues 52 days after transfer of 2×10⁶ T_{H}::iFoxp3 cells (n=4 in each case, for auxiliary lymph node (ax. LN) a pooled sample was analyzed). (D) Summary of the frequency of T_{H}::iFoxp3 cells in the various tissues 17 and 52 days after transfer. (E-H) T_{H}::iFoxp3 cell survival upon 4-OHT withdrawal (E) T_{H}::control and T_{H}::iFoxp3 were cultured in the continuous presence [+>+] or absence [−>−] of 50 nM 4-OHT. In the case of [+>−] 4-OHT was withdrawn for 72 h after an initial induction for 48 h, before their suppressive activity was measured. 1×10⁵ cells of the indicated populations were co-cultured at a 1:1 ratio with 1×10^{5 CD}4⁺CD25⁻ target cells in 96-well plates coated with antiCD3ε [0.6 μg/ml]. The proliferation of the cells was measured after 72 h based on ³H-thymidine incorporation (n=3 in each case). (F-H) T_{H}::control and T_{H}::iFoxp3 were cultured in the presence or absence of 4-OHT [50 nM] and antiCD3ε [0.6 μg/ml]. After 48 h 4-OHT and antiCD3ε was withdrawn. The viability of the cells was assessed by flow cytometry at 0 h, 24 h and 48 h by measuring the co-expression of GFP. (F) Ratio of cells after 4-OHT withdrawal and cells that were cultured in the absence of 4-OHT from the start. (G, H) Representative FACS profiles of T_{H}::control and T_{H}::iFoxp3 cells. All error bars represent the SEM.

FIG. 18. Foxp3 and control retroviral vectors. Diagram of Foxp3 containing retroviral vectors either co-expressing GFP (m6pg[iFoxp3]) or a GPI-linked ratCD8 α-chain (m6p8[iFoxp3]) and retroviral vectors containing a blasticidine-S-deaminase (bsd) as a control gene either co-expressing GFP (m6pg[control]) or a GPI-linked ratCD8 α-chain (m6p8[control]).

FIG. 19. Activation-mediated down regulation of CD62L in T cells. (A, B) CD62L expression on CD4⁺CD25⁻ T_{H} cells (black) and CD4⁺CD25⁺ T_{R} cells (red). (A) Representative FACS profiles for CD62L expression on T_{H} and T_{R} cells prepared from spleen (n=2) and activated with αCD3ε, αCD28 and IL-2 for the indicated length of time. (B) Representative graph of the relative mRNA levels of CD62L in CD4⁺CD25⁻ T_{H} and CD4⁺CD25⁺ T_{R} cells activated for the indicated length of time (n=2) determined by qPCR and normalized to HPRT. (C) Comparison of the horning behavior of activated m6pg[control] transduced CD4⁺CD25⁻ T_{H} (black, n=8) and CD4⁺CD25⁺ T_{R} (red, n=8) cells. 1×10⁶ cells were transferred into each mouse and the tissues were analyzed 48 h later by flow cytometry as described above.

FIG. 20. Adoptive transfer of T_{H}::iFoxp3 cells does not lead to any overt signs of autoimmune disease. Balb/c mice received 2×10⁶ T_{H}::iFoxp3 cells (red, n=7) or no cells (black, n=5) and were visually inspected and weighed weekly for 11 weeks.

FIG. 21. Tamoxifen treatment has no effect on T_{H}::control cells in vivo. Total splenocytes were isolated from mice which had received no transfer of cells or 1×10⁶ polyclonal T_{H}::control and were challenged with ova in CFA. Some of the mice were injected with tamoxifen on day 4 after immunization (n=3 in all cases). The relative proliferation is shown as a ratio of thymidine incorporation in the presence or absence of ova stimulation in the recall reaction performed on day 7. All error bars represent the SEM and the p values were determined using an unpaired t test.

FIG. 22. Level of collagen-specific IgG antibodies. Levels of collagen-specific IgG1, IgG2a, IgG2b and IgG3 on day −2 and 52 in control mice (black, n=6) and mice that had received T_{H}::iFoxp3 cells and tamoxifen injections (red, n=6). All error bars represent the SEM.

FIG. 23. Migration of T_{H}::iFoxp3 cells into the inflamed paw. Mice received either 1×10⁶ T_{H}::iFoxp3 cells or no cell transfer (n=2 in both cases). Arthritis was induced on day 0 by immunization with cII in CFA. The front and hind paws of arthritic mice were dissected on day 45 and the GFP⁺ cells were detected by flow cytometry. Error bars represent the SEM.

FIG. 24. Survival of T_{H}::iFoxp3 cells in the presence or absence of antigen. Mice received 1×10⁶ polyclonal T_{H}::iFoxp3 cells on day 0 and were immunized with ova as indicated on day 5. Some of the mice also received tamoxifen injections either on day 0 or day 8. The number of T_{H}::iFoxp3 cells present in the spleen was assessed by flow cytometry based on GFP expression on day 13. (A) Representative FACS profiles. (B) Summary of the relative number of GFP⁺ cells in the spleen normalized to the total number of recovered cells (n=3 in absence and n=4 in the presence of ova immunization). All error bars represent the SEM.

FIG. 25. In vivo depletion of T_{H}::GFP/TK cells. CD4⁺CD25⁻ T cells were transduced with a retroviral vector containing GFP co-expressing a herpes simplex thymidine kinase gene (m6ptk[GFP]). 24 h after transduction, 1×10⁶ cells were transferred into wild-type mice (day 0). Ganciclovir [1 mg/mouse] was administered for three consecutive days by i.p. injection and on day 5 the inguinal lymph nodes and spleen were analyzed for the presence of T_{H}::GFP/TK cells (n=4 in all cases). All error bars represent the SEM.

FIG. 26 shows graphs.

FIGS. 27 and 28 show plots.

The invention is now described by way of example. These examples are intended to be illustrative, and are not intended to limit the appended claims.

### EXAMPLES

### Methods

Animals and cell preparations. Balb/c and DBA/1 mice (8-12 weeks) were purchased from Charles River, UK and Harlan, UK respectively. Animals were maintained under specific pathogen-free conditions. Cells, used for in vivo and ex vivo experiments were purified (>90% purity) using an AutoMACS (Miltenyi Biotec, UK)¹³. Expert animal technicians provided animal care in compliance with the relevant laws and institutional guidelines. Flow cytometric analysis and proliferation assays were performed as described previously¹³.

Retroviral vectors and transduction. Retroviral transduction was performed as described previously¹³. Six hours after transduction, cells were resuspended in RPMI/10% FCS/10 μM β-mercaptoethanol/10 IU/ml IL2. A fixed ratio of transduced (50-60% in all cases) and non-transduced cells was adoptively transferred into mice after 72 h.

Collagen induced arthritis and gene induction. Male DBA/1 mice received 1-2×10⁶ transduced cells i.v (day −1) and were immunized i.d. with 100 μl chicken Collagen Type II dissolved in 10 mM acetic acid (Sigma) and emulsified [1 μg/μl] in Complete Freund's Adjuvant (DIFCO) the following day (day 0)¹⁹. For iFoxp3 induction the mice were injected i.p. with 100 μl tamoxifen (in 10:1 sunflower oil/ethanol) [10 μg/μl] on days 15 and 16 and [1 μg/μl] on days 23, 29, 30, 36 and 43.

### Example 1

### Cell Homing Behaviour

### Background

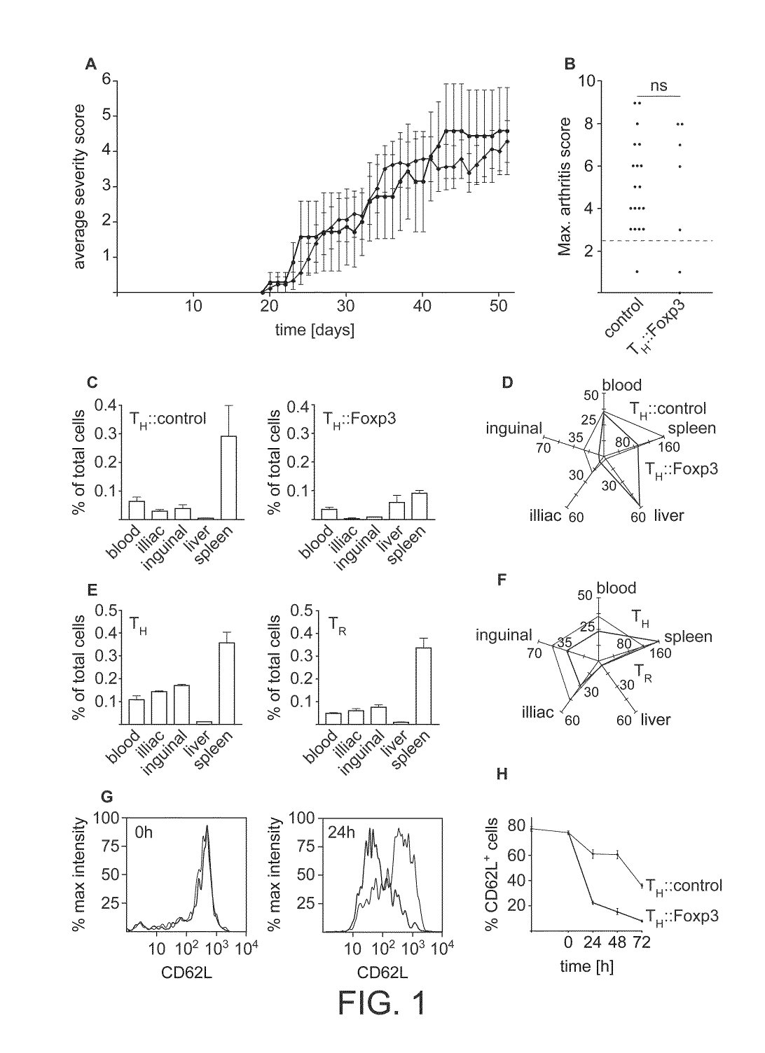

The efficacy of the use of naïve, polyclonal wild type T_{H}::Foxp3 cells to treat autoimmune disease has been very limited^{7,12}. Indeed, our own attempts to treat collagen-induced arthritis with T_{H}::Foxp3 cells, i.e. cells constitutively expressing Foxp3 according to the prior art, failed entirely (FIGS. 1a and b). This might be due to the low frequency of antigen specific cells within the transferred population¹¹. The low number of antigen-specific T_{H}::Foxp3 cells in a polyclonal pool of cells might be overwhelmed by the high number of already expanded pro-inflammatory T cells. However, as we have demonstrated that antigen experienced regulatory T cells are effective suppressors at extremely low ratios¹³, we found this to be an inadequate explanation.

### Homing Behaviour

According to the insight of the inventors, it was suspected that the process of generating T_{H}::Foxp3 cells altered their homing behaviour. Indeed, we find that most of the T_{H}::Foxp3 cells failed to home into the secondary lymphoid organs and instead appeared to accumulate in the liver (FIGS. 1c and d). This is in stark contrast to the cells transduced with an irrelevant control gene, which did not prevent efficient homing of the cells to the secondary lymph nodes and mimicked the homing behaviour of primary cells (FIGS. 1e and f). This observation deserved some closer examination.

### CD62L

CD62L has been described to be one of the key molecules involved in the homing of T cells to the secondary lymphoid organs¹⁴ and it has been shown that only CD62L^{hi} regulatory T cells have a protective effect in vivo¹⁵. It is noteworthy that retroviral transduction requires at least some degree of activation of the cell in order to push them into S-phase of mitosis. We found that in the presence of Foxp3 this lead to a very marked and sustained down-regulation of surface CD62L (FIGS. 1g and h). Whilst we cannot exclude that ectopic expression of Foxp3 alters the expression of further homing receptors, one would expect the change in CD62L surface expression to alter the homing behaviour of the cells¹⁶. This in turn is likely to hinder the T_{H}::Foxp3 cells from mimicking the homing behaviour of regulatory T cells, leading to the low efficacy of these cells in suppressing immune responses in an antigen specific manner¹⁷.

### Example 2

### Inducible Lineage Factors

Next we demonstrate a strategy that utilizes an inducible lineage factor. We demonstrate a method of switching the phenotype of a target cell, which method comprises inducing lineage, factor activity in the target cell via a transgene. In this example the lineage factor is Foxp3 (inducible Foxp3=“iFoxp3”), and the transgene encodes Foxp3 polypeptide having lineage factor activity. In this example the transgene is introduced into the target cell using a retroviral vector.

According to the invention cells transduced with a retroviral transgene expressing iFoxp3 (T_{H}::iFoxp3 cells) should retain the phenotype of pro-inflammatory T cells. When encountering an antigen they should participate in the immune response, expand and exert their pro-inflammatory functions until Foxp3 is induced. Upon induction, the transduced cells should assume the phenotype of regulatory T cells and suppress the response they are involved in. This approach has the advantage that the transduced cells should home normally. This approach has the further advantage that antigen specific cells should ‘self-select’ and expand in the same way as any other cell involved in the response.

### Manufacture of Inducible Lineage Factor Transgene

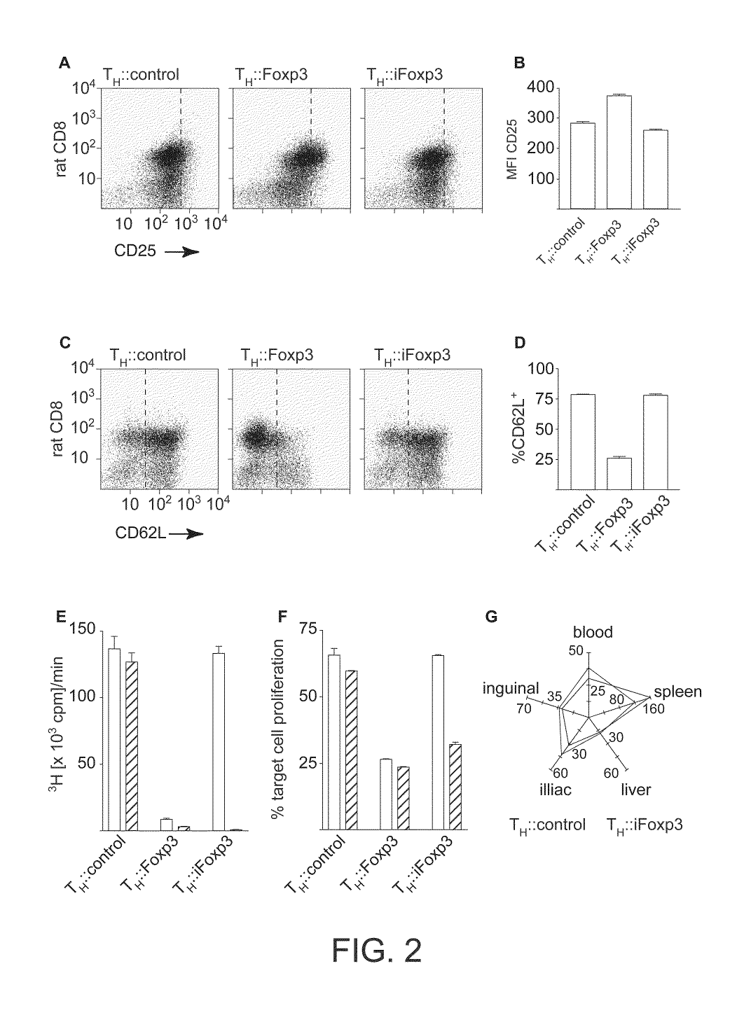

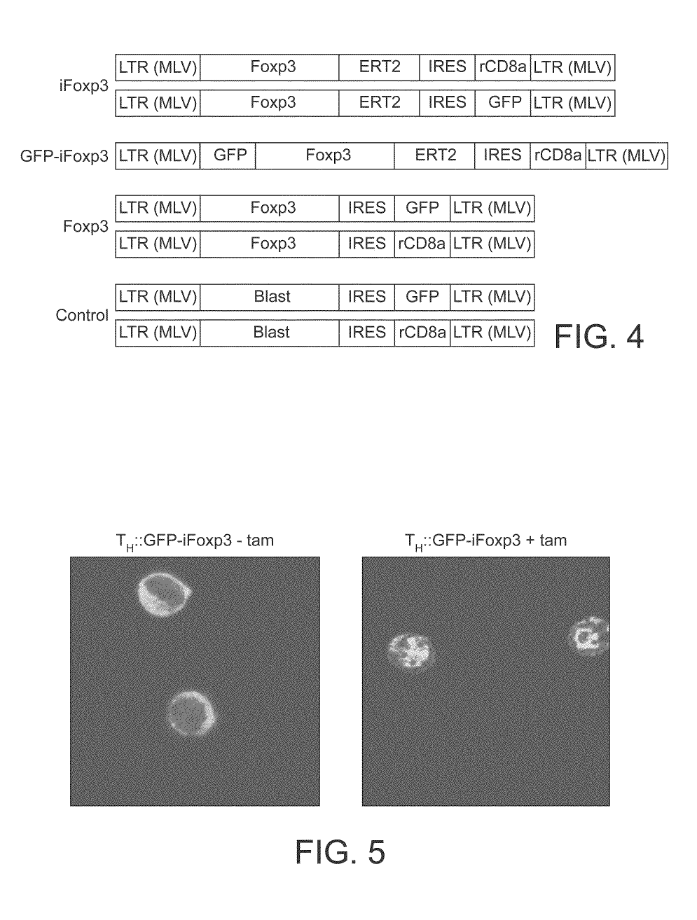

In this example the lineage factor is Foxp3. In this example, the inducibility is provided by control of the subcellular localisation of the lineage factor via fusion to a control polypeptide. Thus, we fused a modified estrogen receptor which only responds to tamoxifen (ERT2)¹⁸ to the C-terminal end of Foxp3 and cloned it into our standard retroviral vector (FIG. 4). The Foxp3ERT2 fusion protein is retained in the cytoplasm by heat shock proteins binding to the ERT2 part of the chimeric protein. As Foxp3 must be in the nucleus to modify the transcriptional program of the cell, it is thereby rendered inactive. In contrast to transduction of the cells with Foxp3, transduction with iFoxp3 resulted neither in a marked increase in CD25 expression beyond that of cells transduced with a control gene (FIGS. 2a and b) nor in down-regulation of CD62L (FIG. 2c and d). However, CD62L surface expression in activated T_{H}::iFoxp3 cells is rapidly down-regulated if iFoxp3 is induced by tamoxifen (FIG. 2e).

### Phenotype Switching

In the absence of induction, T_{H}::iFoxp3 cells appear to retain the phenotype of proinflammatory cells. They are neither anergic (FIG. 2f) nor do they have any suppressive activity (FIG. 2g). Only upon exposure to tamoxifen does the Foxp3ERT2 fusion protein translocate to the nucleus, and the T_{H}::iFoxp3 cells assume regulatory T cell phenotype. They become anergic (FIG. 2f) and gain suppressive activity (FIG. 2g).

To examine the kinetics of the induction process, we coupled a time course of tamoxifen exposure to an in vitro suppression assay (FIG. 2g). Suppression of target cells can be observed if tamoxifen is added at the time of set-up (0 h). However, full suppression activity is only reached if iFoxp3 is induced at least 24 h prior (−24 h) to the use of the cells in the assay. Like T_{H}::control cells, T_{H}::iFoxp3 cells mimic the homing behaviour of primary cells and preferentially accumulate in the secondary lymphoid organs (FIG. 2h). To assess the induction process in vivo we injected cells transduced with a retroviral vector carrying a GFP-tagged iFoxp3 into wild type Balb/c mice. Microscopic analysis of FACSsorted GFP⁺ splenocytes prepared from either tamoxifen or control treated mice confirmed the induction of iFoxp3 in vivo (FIG. 5).

Thus, it is demonstrated that T_{H}::iFoxp3 cells retain their pro-inflammatory phenotype unless they are induced, which in this example is performed by exposure to tamoxifen. Only upon this induction do they switch phenotype and assume the characteristics of regulatory T cells.

### Example 3

### Expansion and Switching of Target Cells Using Inducible Lineage Factors

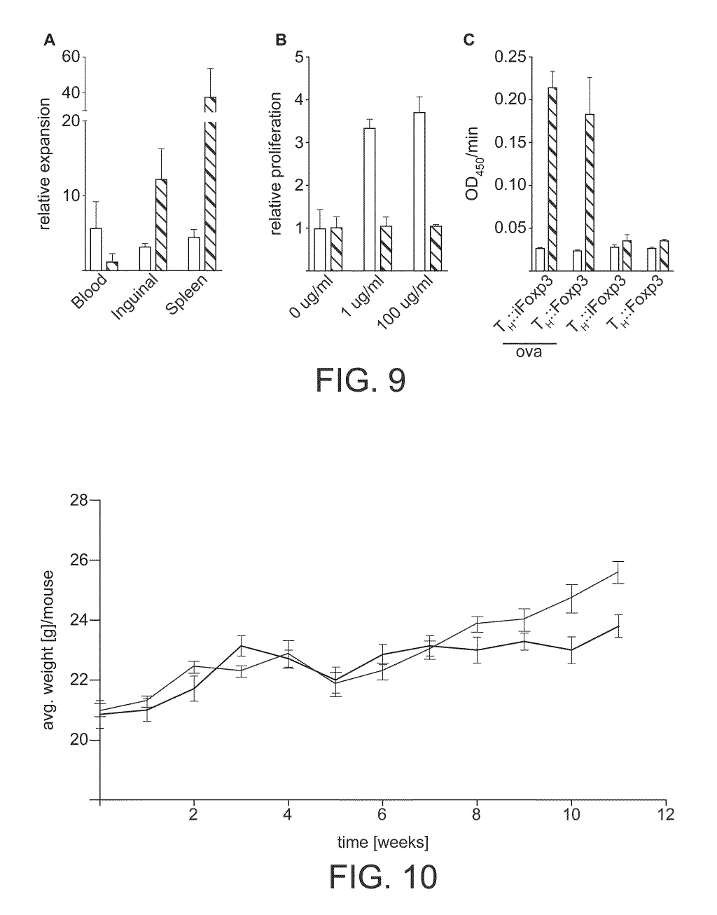

To assess whether T_{H}::Foxp3 and T_{H}::iFoxp3 cells expand upon antigenic challenge in vivo, we transferred Foxp3− or iFoxp3-transduced T cells from DO11.10xSCID mice, expressing an ovalbumin-specific T cell receptor transgene, into wild type Balb/c mice. In order to approximate physiological conditions whilst still retaining a measurable effect, we transferred only 2×10⁴ cells transduced cells (19). We found that T_{H}::iFoxp3 cells expanded upon immunization with ovalbumin (ova) by a factor of 12 in the draining lymph nodes and a factor of 37.5 in the spleen. In contrast, T_{H}::Foxp3 cells only exhibited a very modest expansion by a factor of 3.6 in the lymph nodes and 4.4 in the spleen (FIG. 9A). This could have been due to the T_{H}::Foxp3 cells limiting the response and thereby impeding their own expansion. However, when we examined the levels of ova specific antibodies in the serum, we found no difference between mice having received T_{H}::Foxp3 or T_{H}::iFoxp3 cells, suggesting this was not the case (FIG. 9B). Our data demonstrates a clear expansion of T_{H}::iFoxp3 cells, which is consistent with their participation in the immune response against ova.

Next we investigated whether the in vivo expanded ova specific T_{H}::iFoxp3 cells can be induced to suppress the very same immune response they partake in. We isolated the splenocytes from these mice and exposed them to ova in vitro. Whilst in the absence of induction we observed the expected antigen-induced recall proliferation, we could not detect any proliferation above background in the presence of tam (FIG. 9C). This suggests that upon induction the T_{H}::iFoxp3 cells assumed a T_{R} cell phenotype and suppressed the proliferation of both the endogenous ova-specific T cells as well as the co-transferred non-transduced DO11.10 T cells.

### Example 4

### Suppression of Immune Responses

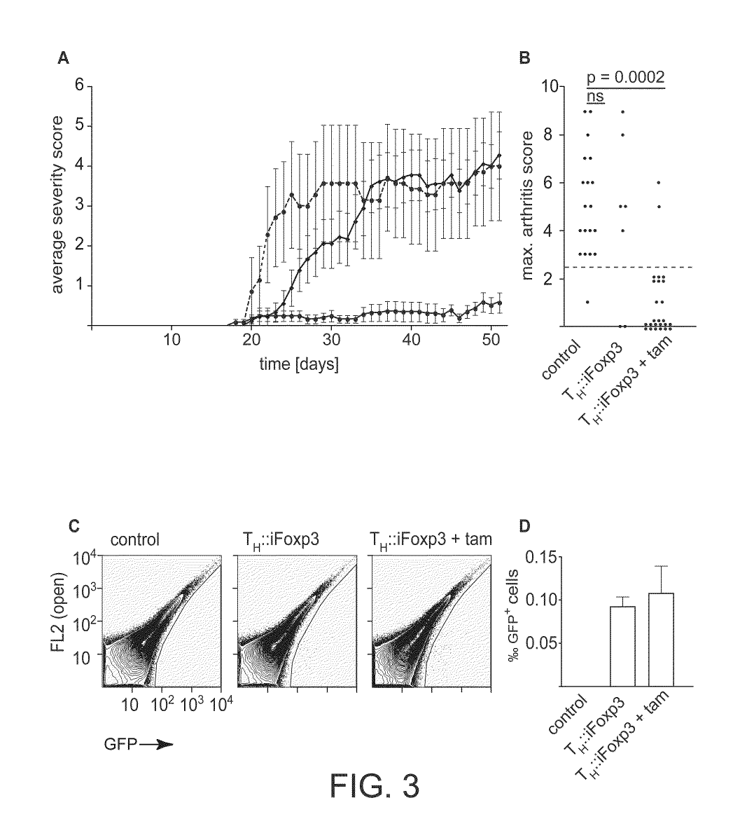

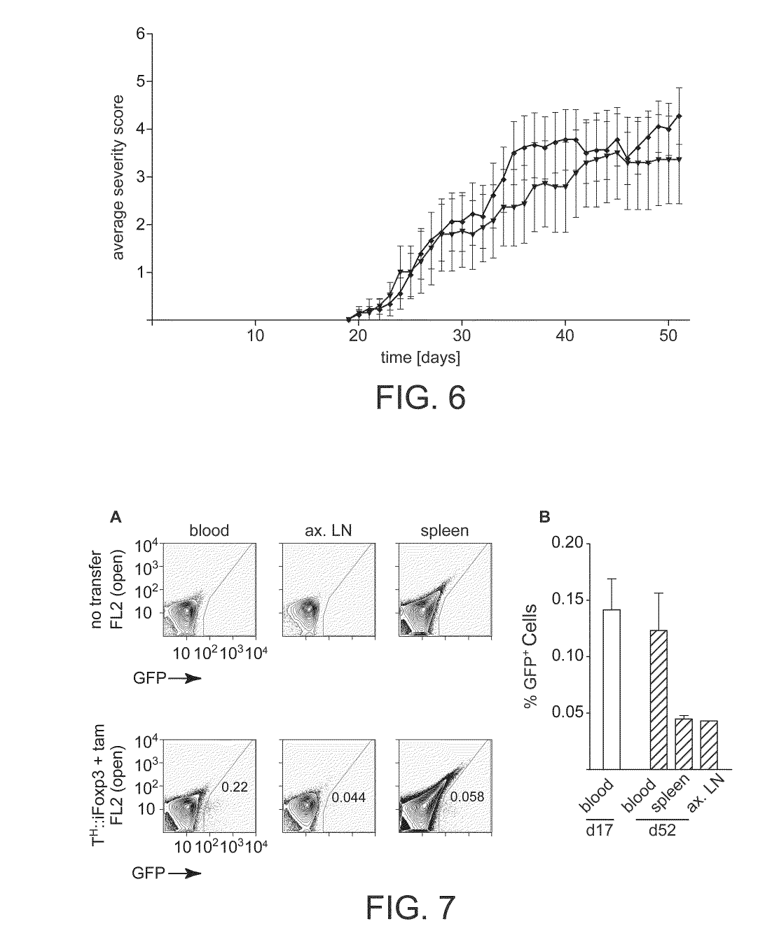

Following from example 3, in order to demonstrate the efficacy in suppressing immune responses in vivo, we turned to a collagen-induced arthritis model. Arthritis was induced by immunization of male DBA/1 mice with chicken collagen type II in Complete Freund's Adjuvant. Adoptive transfer of T_{H}::iFoxp3 cells was performed one day prior (day −1) to immunization (day 0). Induction of iFoxp3 was achieved by injections of tamoxifen from day 15 onwards. Arthritis was scored blindly on a daily basis according to a standardized scoring system¹⁹ (FIG. 3a and b). In the control group, first signs of arthritis were observed on day 18 and a plateau was reached at around day 35. Mice that had received T_{H}::iFoxp3 cells, but which did not receive tamoxifen injections also showed first signs of arthritis on day 18. However, the onset of arthritis in this group was more marked. In this case a plateau was reached a week earlier on day 28. The average arthritis score on reaching the plateau was the same for both groups. Remarkably, 23 out 25 of the mice, which had received TH::iFoxp3 cells and tamoxifen injections, did not show any clear signs of arthritis. Whilst tamoxifen itself has been reported to have anti-inflammatory properties²⁰, we found that it had only a mild effect, if any, on the development of collagen-induced arthritis in the absence of T_{H}::iFoxp3 cells (FIG. 6). Interestingly, we were able to detect T_{H}::iFoxp3 cells 52 days after their transfer, independent of the level of arthritis and whether the mice received tamoxifen treatment or not (FIG. 3c,d and FIG. 7).

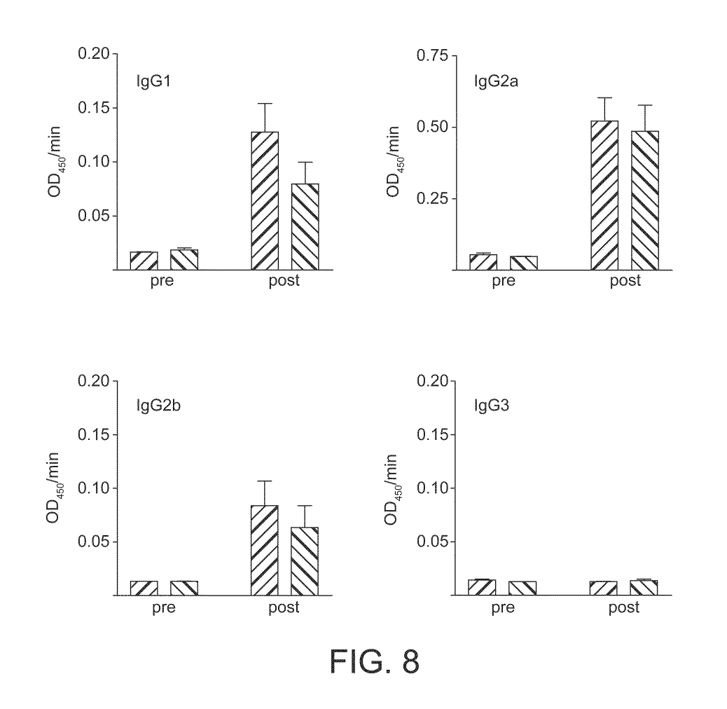

This demonstrates that T_{H}::iFoxp3 cells are present throughout, but do not suppress the response in the absence of induction. The fact that the level of anti-collagen IgG antibodies detected in mice in which iFoxp3 was induced and in control mice that developed arthritis were similar (FIG. 8), shows that we are indeed stopping an ongoing response rather than merely preventing its onset. By the time iFoxp3 is induced, the anti-collagen antibody response is already well advanced. Nevertheless, the induction of T_{H}::iFoxp3 cells was successful in completely stopping arthritis in over 90% of the cases.

### Example 5

### Specific Immunosuppression with Inducible Lineage Factor-Transduced Polyclonal T Cells

We show suppression of immune responses with inducible lineage factor; in this example the lineage factor is Foxp3.

### Overview

Foxp3-expressing regulatory. T cells are key mediators of peripheral tolerance suppressing undesirable immune responses. Ectopic expression of Foxp3 confers regulatory T cell phenotype to conventional T cells, lending itself to therapeutic use in the prevention of autoimmunity and transplant rejection. Here, we show that adoptive transfer of polyclonal, wild-type T cells transduced with an inducible form of Foxp3 (iFoxp3) can be used to suppress immune responses on demand. In contrast to Foxp3-, iFoxp3-transduced cells home ‘correctly’ into secondary lymphoid organs, where they expand and participate in immune responses. Upon induction of iFoxp3 the cells assume regulatory T cell phenotype and start to suppress the response they initially partook in without causing systemic immunosuppression. We demonstrate this approach to suppress collagen-induced arthritis, where conventional Foxp3-transduced cells failed to show any effect. This provides with a generally applicable strategy to specifically halt immune responses on demand without prior knowledge of the antigens involved.

### Materials And Methods

Animals and cell preparations. Balb/c and DBA/1 mice (8-12 weeks) were purchased from Charles River (UK) and Harlan (UK). DO11.10xSCID mice on the Balb/c background were kindly provided by Caetano Reis e Sousa, CRUK. Animals were maintained under specific pathogen-free conditions. Expert animal technicians provided animal care in compliance with the relevant laws and institutional guidelines. Cells used for in vivo and ex vivo experiments were purified (>90% purity) using an AutoMACS (Miltenyi Biotec, UK) as previously described [66]. Flow cytometric analysis and proliferation assays were performed as described previously [66] using the following antibodies: ratCD8α (BD Bioscience, UK), CD62L (BD Bioscience, UK), CD4 (BD Bioscience, UK), CD25 (BD Bioscience, UK) and Foxp3 (eBioscience, USA).

Retroviral vectors and transduction. Foxp3 was amplified from total spleen cDNA and iFoxp3 was constructed by a C-terminal fusion of ERT2 in place of the stop codon. Both were cloned into m6p retroviral vectors co-expressing either GFP or a GPI-linked rat CD8α marker. For the measurement of in vivo translocation of iFoxp3, GFP was cloned in-frame with Foxp3 after the first five codons in the 5′-prime-end [67] in order to produce GFP-iFoxp3. For the production of retroviral supernatant, 293eT cells were co-transfected with an equal amount of pCl-Eco packaging plasmid and the respective m6p retroviral construct. Supernatant was harvested at 36 h and 48 h after transfection, filtered and used immediately. For retroviral transduction the freshly purified CD4⁺CD25⁻ T cells were activated in the presence of plate-bound antiCD3ε [0.6 μg/ml] (BD Bioscience, UK) and 10 U/ml of recombinant mIL-2 (PeproTech, UK). Cells were transduced at 24 h and 36 h after activation by re-suspension-in a 1:2 mixture of supernatant and complete medium (RPMI/10% FCS/10 μM β-mercaptoethanol/50 μg/ml gentamicin) supplemented with 10 U mIL-2 and 6 μg/ml Protamine Sulphate (Sigma, UK) and 10 U/ml mIL2, followed by centrifugation at 600×g for 2 h at 32° C. Six hours after transduction, cells were resuspended in complete medium containing 10U mIL-2. A fixed ratio of transduced (50-60% in all cases) and non-transduced cells was adoptively transferred into mice 72 h after the last transduction.

Collagen induced arthritis and gene induction. Male DBA/1 mice received 1−2×10⁶ transduced cells i.v (day −1) and were immunized i.d, with 100 μl cII (Sigma, UK) dissolved in 10 mM acetic acid and emulsified [1 μg/μl] in CFA (DIFCO, USA) the following day (day 0) [46]. The mice were assessed (blinded) on a daily basis and inflammation of the paws was scored as follows: grade 0—no swelling; grade 1—swelling in an individual joint; grade 2—swelling in more than one joint or mild inflammation of the paw; grade 3—severe swelling of the entire paw and/or ankylosis. Each paw was graded and all scores where totaled for a maximum score of 12 per mouse. Mice reaching a score of 8 or more were euthanized in accordance with restrictions imposed by UK legislation. For iFoxp3 induction the mice were injected i.p. with 100 μl tamoxifen (in 10:1 sunflower oil/ethanol) [10 μg/μl] on days 15 and 16 and [1 μg/μl] on days 23, 29, 30, 36 and 43. Alternatively, iFoxp3 was induced once the mice had reached a score of ‘3’ (day 0) by i.p. injections with 100 μl tamoxifen (in 10:1 sunflower oil/ethanol) [10 μg/μl] on days 1, 2, 9 and 16.

In vivo expansion of antigen-specific T cells and ova-specific suppression assay. CD4⁺CD25⁻ T cells were purified from 6-12 week old female SCIDxDO11.10 mice and transduced with Foxp3 or iFoxp3 as described above. Balb/c females received i.v. 5×10⁴ of a 2:3 ratio of transduced and non-transduced cells. Three days later each mouse was immunized s.c. with either ova (Sigma, UK) in CFA [50 μg/mouse] or just with CFA. The mice were sacrificed and analyzed eight days after immunization. For ova-specific suppression assays total splenocytes were prepared as described [66], resuspended in complete medium and plated into round-bottom 96-well plates (density of 2×10⁵ cells/well). iFoxp3 was induced by adding 50 nM 4-OHT (Sigma, UK). Ova was added to the cells 16 h after induction. After 60 h, the cells were pulsed with 1 μCi ³H-thymidine (Amersham, UK), collected at 72 h with a Filtermate Harvester (Packard) and analyzed with a TopCount scintillation counter (Packard) according to the manufacturer's instructions.

Collagen and ova-specific ex vivo recall reactions. CIA and iFoxp3 induction was performed as described above. On day 28, some of the mice received ova in CFA s.c. into both flanks [100 μg/mouse]. Total splenocytes were prepared on day 35 and plated into round-bottom 96-well plates at a density of 5×10⁵ cells/well. Proliferation of the cells was measured 72 h after addition of either ova [100 μg/ml] or cII [100 μg/ml] as described above. Alternatively, mice were immunized simultaneously with ova and cII on day 0 by i.d. injection of a mixture of 100 μg ova and 100 μg cII in CFA. Recall reactions were performed on day 28 as described above at a density of 2×10⁵ cells/well.

Elisa for the detection of collagen and ova-specific antibodies. 96-well flat-bottom plates (Nunc, DK) were coated with either ova [50 μg/ml] or cII [2 μg/ml] at 4° C. for 16 h and blocked with 1% BSA in PBS for 1 h. 50 μl of serial dilutions (starting at 1:50 for ova and 1:10,000 for cII) of mouse sera in PBS were incubated for 2 h. Biotin-conjugated IgG1, IgG2a, IgG2b and IgG3 (BD Bioscience, UK) were then applied for 2 h. For ova detection IgM (BD Bioscience, UK) was also included. The development of cII and ova-specific immunoglobulins was then measured using a DuoSet kit (R&D Systems, UK) according to the manufacturer's instructions.

Real-time RT-PCR. Total RNA was extracted using an RNeasy kit (Qiagen, UK) including DNaseI treatment (Invitrogen, UK). cDNA was synthesized with Superscript II (Invitrogen, UK) with random hexamer primers (Amersham, UK) following the manufacturers instructions. Real-time PCR was performed using Taqman SYBR green PCR master mix (Applied Biosystems, UK) with primers specific for Sell (CD62L) and Hprt. The sequences used were: Sell primers: 5′-ATG CAG TCC ATG GTA CCC AAC TCA-3′ and 5′-CTG CAG AAA CAC AGT GTG GAG CAT-3′; Hprt primers: 5′-TTA AGC AGT ACA GCC CCA AAA TG-3′ and 5′-CAA ACT TGT CTG GAA TTT CAA ATC C-3′. An ABI Prism 7900 sequence detection system (Applied Biosystems, UK) was used for 45 cycles of PCR according to the manufacturer's instructions.

### Introduction

Transplant rejection and autoimmune diseases ranging from Rheumatoid Arthritis, Type I Diabetes, Multiple Sclerosis to Inflammatory Bowel Disease—as diverse as they might appear—all have the same underlying problem: the launch of an undesirable immune response [1]. Equally similar are the current approaches to treat these conditions, which are generally based on drugs that lead to systemic immunosuppression [2]. Thus, the induction of specific tolerance is seen as the ‘Holy Grail’ of therapeutic approaches [3].

The discovery that the immune system evolved regulatory T (T_{R}) cells to stop undesirable immune responses, such as autoimmunity [4] and the rejection of the fetus [5-7], is of obvious therapeutic promise [8]. Indeed, T_{R} cells have already been shown to be capable of fulfilling such functions [9]. However, the translation of experimental findings into actual therapeutic approaches is hampered by a variety of problems. Under experimental conditions, antigen-specific tolerance can be achieved by using T_{R} cells from TCR-transgenic animals or by ex vivo expansion of antigen-specific T_{R} cells [9-11]. However, it is difficult to imagine how a TCR transgenic approach can be translated into a generally applicable therapy. The antigen-specific ex vivo expansion of T_{R} cells [9-11], or in vivo conversion of T_{H} into T_{R} cells [12], are more feasible, albeit still problematic. They not only rely on the knowledge of, or at least access to the antigens involved in the pathological immune response, but are also time consuming and complicated when applied in a therapeutic context [8,13].

There are also conceptual problems. The lack or malfunction of T_{R} cells is suspected to be at the root of many autoimmune diseases [14,15]. In these cases, it might be impossible to obtain and expand functional, antigen-specific T_{R} cells, as they may not exist in the host in the first place. In principle, this problem can be circumvented by the conversion of conventional T cells into T_{R} cells, either by TGF-β mediated induction [16-18] or ectopic expression of the lineage factor Foxp3 (NP_{—}473380) [19-21]. However, without enriching antigen-specific ‘induced T_{R} cells’ this is likely to be of limited benefit and may lead to systemic immune-suppression [11,22-24]. A further problem with TGF-β induced T_{R} cells is that their phenotype seems to be unstable [25,26], although the presence of retinoic acid appears to stabilize the conversion [27,28].

By contrast, the invention provides a strategy to suppress undesirable immune responses in an antigen-specific fashion without prior knowledge of the antigens involved. We accomplish this by adoptive transfer of a small number of polyclonal T_{H} cells transduced with a genetically engineered, inducible form of lineage factor (in this example the lineage factor is Foxp3) (iFoxp3). CD4⁺CD25⁻ cells transduced with iFoxp3 (T_{H}::iFoxp3) initially retain their ‘pro-inflammatory’ phenotype. They home ‘correctly’ into the secondary lymphoid organs and partake in immune responses. Once the T_{H}::iFoxp3 cells have expanded in an antigen-specific fashion they can be converted to T_{R} cell phenotype on demand by inducing iFoxp3, thereby stopping the immune response they partook in.

### Failure of Polyclonal T_{H}::Foxp3 Cells to Suppress CIA

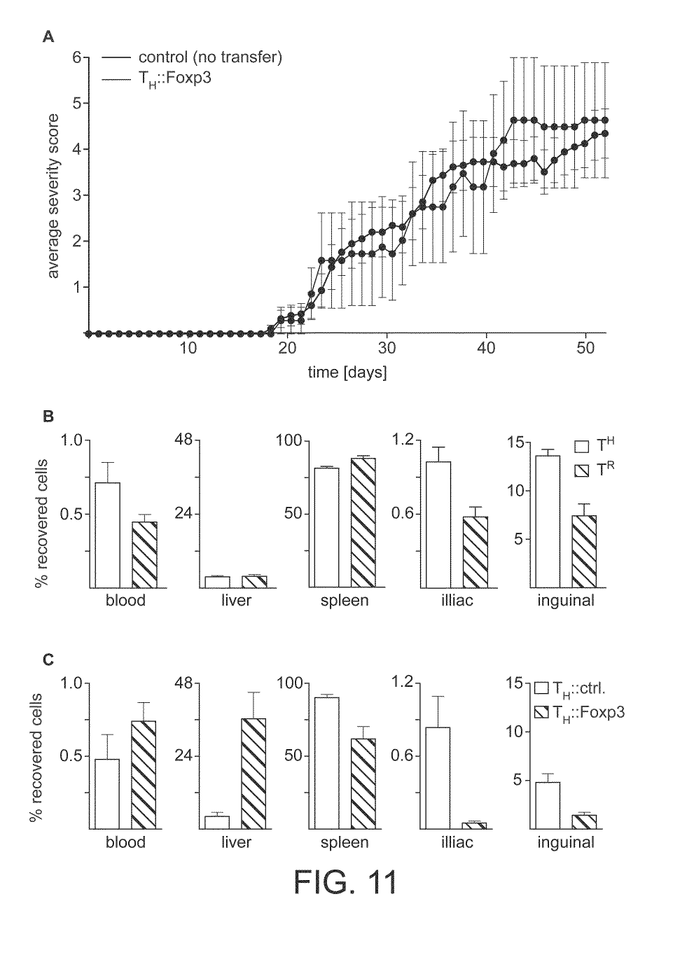

Encouraged by the initial finding that polyclonal CD4⁺CD25⁻ T cells transduced with Foxp3 (T_{H}::Foxp3) can prevent and treat colitis in lymphopenic animals [19,29] we, like others [23,30,31], set out to test whether this can be used as a general strategy to prevent and treat autoimmune diseases. To test this hypothesis, we used collagen-induced arthritis (CIA), which is a well-established murine model of human rheumatoid arthritis [32]. To obtain T_{H}::Foxp3 cells, we transduced CD4⁺CD25⁻ T cells with a MLV-based retroviral vector carrying a Foxp3-IRES-GFP cassette (m6pg[Foxp3]) (FIG. 18). We immunized male DBA/1 mice with chicken collagen type II (cII) in Complete Freund's Adjuvant (CFA). In this model, we observe the first clinical symptoms of arthritis on day 19 after immunization, with the average clinical score reaching a plateau around day 35. Injection of 1×10⁶ T_{H}::Foxp3 cells one day prior to immunization did not have any significant impact on the outcome of the arthritis. It neither delayed the time of disease onset, nor did it alter disease progression (FIG. 11A). The failure of polyclonal T_{H}::Foxp3 cells to show any beneficial effect on the outcome of CIA under these experimental conditions, is in agreement with the findings of others [31] and led us to reassess the approach per se. Therefore, we decided to examine the homing, expansion and participation of T_{H}::Foxp3 cells in immune responses.

### Altered Homing Behavior of T_{H}::Foxp3 Cells

The decision whether to launch or suppress an immune response is made within the secondary lymphoid organs [33]. This makes ‘correct’ homing of the adoptively transferred cells an essential requirement for cyto-therapy, as otherwise their participation in immune responses might be severely limited.

We therefore compared the homing of T_{H}::Foxp3 cells to that of m6 pg[control] transduced CD4⁺CD25⁻ T (T_{H}::control) cells (FIG. 18) and freshly isolated CFSE labeled CD4⁺CD25⁻ (T_{H}) cells or CD4⁺CD25⁺ (T_{R}) cells. 1×10⁶ cells were injected into wild type Balb/c mice. After 48 h, we isolated the lymphocytes from the various tissues and analyzed them by flow cytometry. The transferred cells were identified based on either their GFP co-expression or CFSE label. T_{H}::control cells, like T_{R} and T_{H} cells could be detected at comparable frequencies in blood, inguinal and iliac lymph nodes, as well as the spleen (FIGS. 11B and C). In contrast, the homing of T_{H}::Foxp3 cells into the lymph nodes appeared to be defective and their homing into the spleen slightly impaired. Instead, a large number of these cells could be found in the liver (FIG. 11C). The data suggest that ectopic expression of Foxp3 substantially altered the homing behavior of the transduced cells.

### Foxp3 Mediated Regulation of CD62L

The absence of T cells from the peripheral lymph nodes is one of the key features of CD62L-deficient (sell^{-/-}) mice [34]. CD62L (L-selectin) plays a key role in the homing of lymphocytes into these tissues by allowing their attachment to high endothelial venules [35]. Activation of T cells leads to endoproteolytic shedding of CD62L from the surface of the cells, involving the matrix-metalloprotease Adam17 [36]. Therefore, we investigated whether the altered homing behavior of T_{H}::Foxp3 cells is due to Foxp3-mediated effects on the surface expression of CD62L.

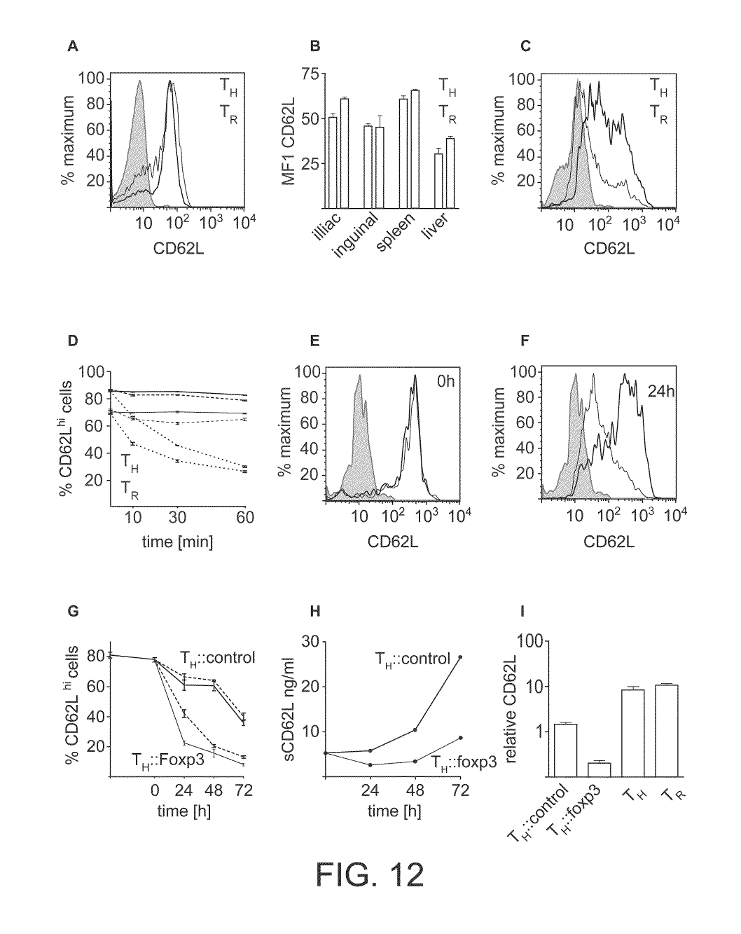

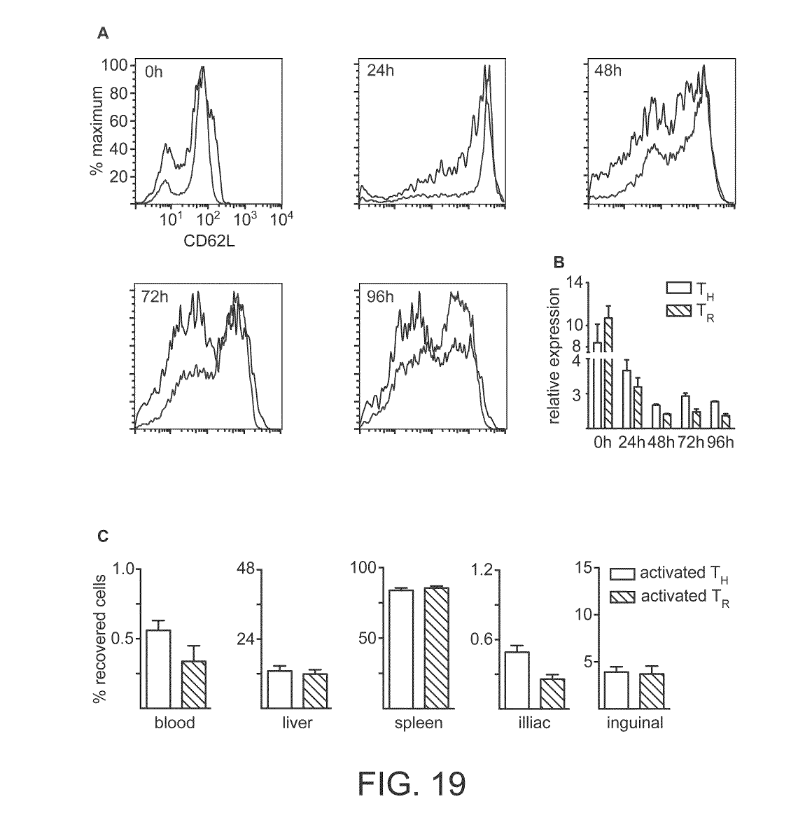

We found that the majority of freshly isolated T_{H} and T_{R} cells are CD62L^{hi} (FIGS. 12A and B). Activation of the cells for 72 h with antiCD3/antiCD28/IL-2 led to a down-regulation of CD62L surface expression, which was more marked in T_{R} than T_{H} cells (FIGS. 12C and 19A). To assess whether this is due to an increase in Adam17 activity in T_{R} cells, we activated freshly isolated splenocytes with PMA and compared the surface expression of CD62L on Foxp3⁺ (T_{R}) and Foxp3⁻ (T_{H}) CD4⁺ T cells. The rate of CD62L shedding appeared to be very similar for both cell types and could be completely blocked by the Adam17 inhibitor TAPI-2 (FIG. 12D). This suggests, that an additional Adam17-independent mechanism in T_{R} cells is responsible for the difference in CD62L surface expression observed upon activation of T_{R} and T_{H} cells.

To further investigate this, we examined CD62L expression in T_{H}::Foxp3 cells. We transduced CD4⁺CD25⁻ cells with either m6p8[Foxp3] or m6p8[control]. The cells carrying the vector were identified based on their co-expression of ratCD8α (FIG. 18). Whilst T_{H}::control cells exhibited some down-regulation of surface CD62L upon activation with antiCD3/IL-2, this was substantially more marked in T_{H}::Foxp3 cells (FIGS. 12E and F). For the first 24 h, TAPI-2 appeared to partially inhibit the loss of surface CD62L on T_{H}::Foxp3 cells, but it did not halt the steady decrease in surface CD62L over an extended period of time (FIG. 12G). The CD62L down-regulation in T_{H}::control cells was accompanied by an accumulation of soluble CD62L in the culture supernatant. This was not the case for T_{H}::Foxp3 cells (FIG. 12H), suggesting that in these cells CD62L surface expression is regulated by a mechanism other than shedding. As Foxp3 is known to be a transcriptional regulator [37-40], we investigated whether it affects CD62L transcription. The CD62L mRNA expression level was reduced in both T_{H}::Foxp3 and T_{H}::control cells compared to freshly isolated T_{H} and T_{R} cells (FIG. 12I). However, the level of CD62L transcript was 7.2 fold lower in T_{H}::Foxp3 cells than in T_{H}::control cells. The data suggest that upon activation of the cells, CD62L is further down-regulated on a transcriptional level by Foxp3.

It is noteworthy, that retroviral transduction requires at least some degree of activation of the cell to allow for transgene integration. In this context, the expression of Foxp3 led to a very marked and sustained down-regulation of surface CD62L expression. This is likely to be a major contributor to the altered homing behavior of T_{H}::Foxp3 cells. Whilst the down-regulation of CD62L upon activation is similarly more evident in thymically derived T_{R} cells than T_{H} cells (FIGS. 19A and B), albeit less marked than in T_{H}::Foxp3 (FIG. 12I), it does not appear to interfere with the cells ability to home into peripheral lymph nodes (FIG. 19C).

iFoxp3—an Engineered Inducible Lineage Factor

The ‘incorrect’ homing of polyclonal T_{H}::Foxp3 cells might well contribute to their lack of showing any beneficial effect in CIA [31] (FIG. 11A) and other animal models of autoimmune disease [11]. However, one might question whether our initial approach had any merit in the first place, since the transfer of polyclonal T_{H}::Foxp3 cells will only marginally increase the number of suppressive cells that recognize a particular antigen. Indeed, treatment with polyclonal T_{H}::Foxp3 cells more or less mimics polyclonal T_{R} cell therapy, which in contrast to approaches using antigen-specific T_{R} cells, appears to be of limited benefit [22-24,41].

We decided to develop an alternative strategy, allowing us to convert the lineage commitment of conventional T_{H} cells to that of T_{R} cells after their antigen-specific expansion in vivo. To achieve this, we created an inducible Foxp3 (iFoxp3) that is constitutively expressed, but only becomes functionally active upon induction. Polyclonal, primary T_{H} cells transduced with iFoxp3 (T_{H}::iFoxp3 cells) should act like conventional T cells, retain their homing behavior, participate in immune responses and expand in an antigen-specific fashion. This antigen-specific in vivo expansion of T_{H}::iFoxp3 cells should allow us to specifically switch off immune responses on demand by inducing iFoxp3.

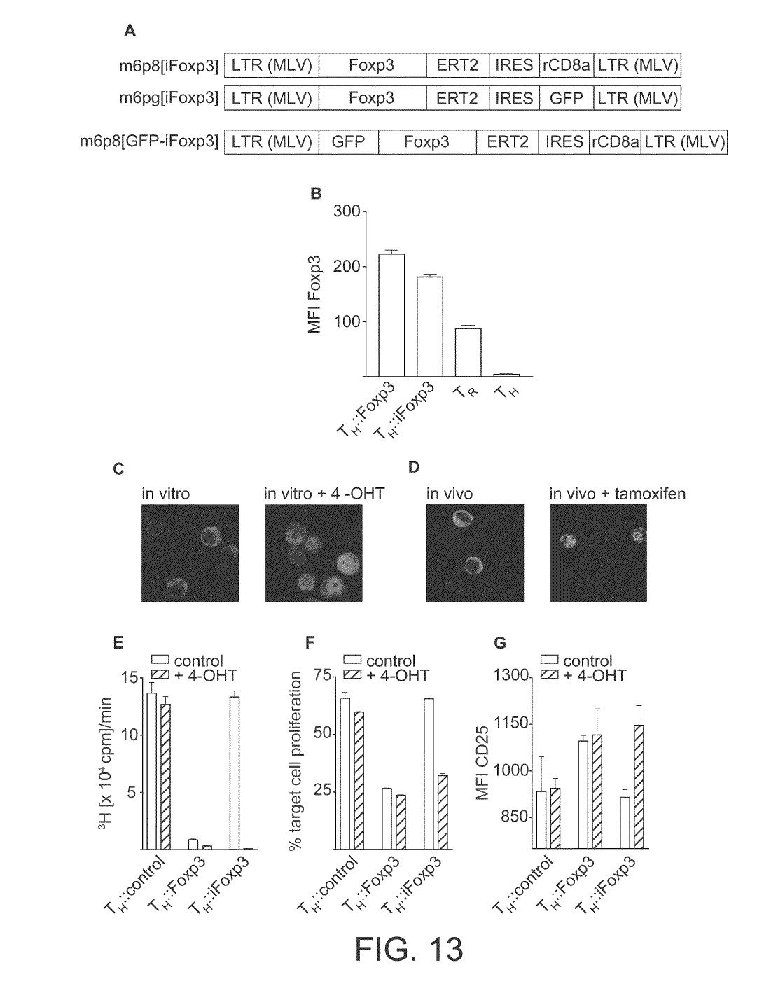

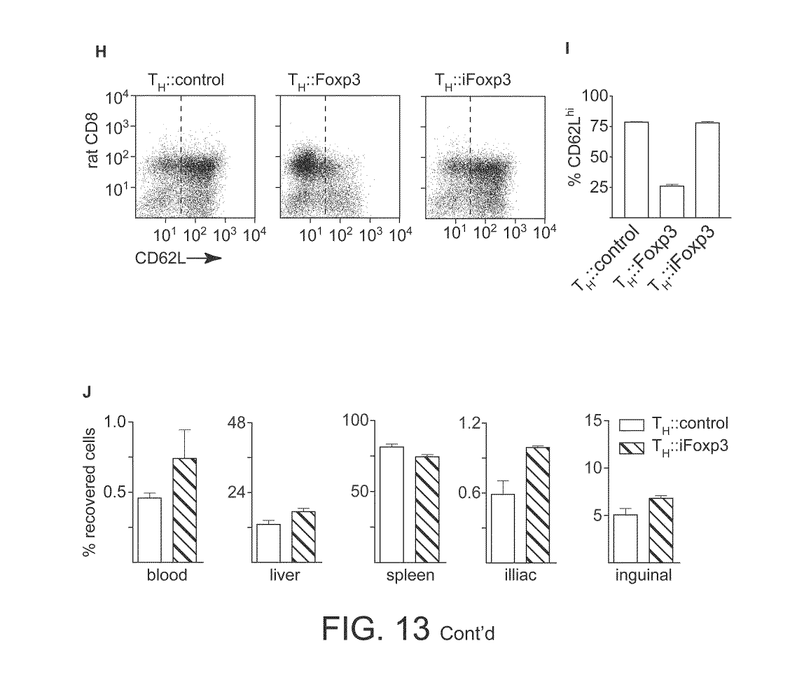

We fused a modified estrogen receptor (ERT2) to the C-terminal end of Foxp3 and cloned it into the m6p vector (FIGS. 13A and B). ERT2 only responds to tamoxifen and its metabolites such as 4-hydroxytamoxifen (4-OHT), but not estrogen [42]. In the absence of induction, iFoxp3 is retained in the cytoplasm and kept inactive by heat shock proteins binding to the ERT2 part of the fusion protein [43]. To confirm the inducible nature of iFoxp3, we transduced CD4⁺CD25⁻ cells with m6p carrying a GFP-tagged iFoxp3 (m6p8[GFP-iFoxp3]). This allowed us to assess the induction of iFoxp3 based on the translocation of the fusion protein from the cytoplasm into the nucleus. We induced iFoxp3 in vitro by exposure to 4-OHT for 48 h (FIG. 13C) or in vivo after adoptive transfer of the transduced cells into wild type Balb/c mice by i.p. injections of tamoxifen (FIG. 13D). In either case, iFoxp3 translocated into the nucleus in about 60-70% of the transduced cells at the time of microscopic analysis, confirming its inducible nature.

Induction of Suppressor Function in T_{H}::iFoxp3 Cells

A key requirement for our strategy is that iFoxp3 can be used to induce T_{R} cell phenotype on demand. We therefore tested T_{H}::iFoxp3 cells for hallmark features of T_{R} cells such as sustained up-regulation of CD25, in vitro anergy to antiCD3-stimulation and suppression of target cells [4] before and after induction of iFoxp3. Whereas T_{H}::Foxp3 cells were anergic (FIG. 13E), suppressed the proliferation of co-cultured CD4⁺CD25⁻ cells (FIG. 13F) and exhibited up-regulation of CD25 (FIG. 13G), T_{H}::iFoxp3 cells did so only after induction of iFoxp3 with 4-OHT. This demonstrates that, at least in vitro, T_{H}::iFoxp3 cells appear to behave like conventional T_{H} cells and only assume the phenotype of T_{R} cells upon the induction of iFoxp3.

T_{H}::iFoxp3 Home Like Naive CD4⁺T Cells

From our observations with T_{H}::control cells, we already knew that transduction per se did not appear to alter the homing behavior of the cells (FIGS. 11B and C). Nevertheless, we wanted to verify that non-induced iFoxp3 neither changes the expression of CD62L, nor significantly alters the homing behavior of the T_{H}::iFoxp3 cells. We found that in the absence of iFoxp3 induction, CD62L expression remained unchanged in T_{H}::iFoxp3 compared to T_{H}::control cells (FIGS. 13H and I). This is in stark contrast to our observations made for T_{H}::Foxp3 cells (FIGS. 12E to I). To assess the homing behavior of the cells we used the same approach as described above. We found that the homing behavior of T_{H}::iFoxp3 cells was comparable to that of T_{H}::control cells (FIG. 13J) and thus very similar to that of naive T_{H} and T_{R} cells (FIG. 11B).

Antigen-Specific In Vivo Expansion of T_{H}::iFoxp3 Cells

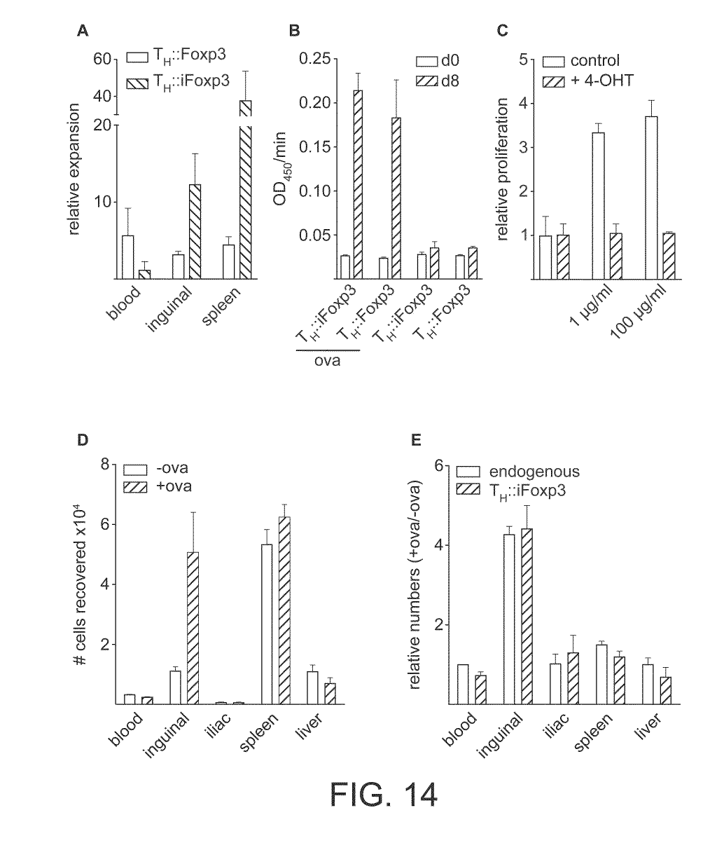

To assess whether T_{H}::Foxp3 and T_{H}::iFoxp3 cells expand upon antigenic challenge in vivo, we transferred transduced cells prepared from DO11.10xSCID/Balb/c mice that expressed an ovalbumin (ova) specific TCR, into wild-type Balb/c mice. We transferred 5×10⁴ cells containing a mixture of 2×10⁴ T_{H}::iFoxp3 cells and 3×10⁴ non-transduced cells (transduction efficiency of 40%) with the transduced population being clearly identifiable based on the co-expression of GFP. T_{H}::iFoxp3 cells expanded upon immunization with ova in CFA by a factor of 12 in the draining lymph nodes and by a factor of 37.5 in the spleen (FIG. 14A). In contrast, T_{H}::Foxp3 cells only exhibited a very modest expansion by a factor of 3.6 in the lymph nodes and 4.4 in the spleen. This could have been due to the T_{H}::Foxp3 cells suppressing the ova-specific immune response and thereby impeding their own expansion. However, the levels of ova-specific antibodies in the serum were the same, independent of whether the mice had received T_{H}::Foxp3 or T_{H}::iFoxp3 cells, suggesting this was not the case (FIG. 14B). Our data demonstrates a clear expansion of T_{H}::iFoxp3 cells, which is consistent with their participation in the immune response against ova. This in vivo expansion upon antigen exposure is considerably less marked in T_{H}::Foxp3 cells.

Next, we investigated whether the in vivo expanded ova-specific T_{H}::iFoxp3 cells can be induced to suppress the very same immune response they partook in. We isolated splenocytes from these mice and exposed them to ova ex vivo. Whilst in the absence of induction of iFoxp3 we observed the expected antigen-induced recall proliferation, we could not detect any proliferation above background in the presence of 4-OHT (FIG. 14C). This suggests that upon iFoxp3 induction the expanded T_{H}::iFoxp3 cells became anergic and suppressed the proliferation of the co-transferred, non-transduced DO11.10 T cells as well as any endogenous ova-specific T cells.

To assess to what degree polyclonal T_{H}::iFoxp3 participate in an immune response, we transferred 1×10⁶ wild-type T_{H}::iFoxp3 cells into wild-type Balb/c mice. A week after immunization with ova, we analyzed the lymphocytes from various tissues by flow cytometry. Whilst the number of T_{H}::iFoxp3 cells recovered from the blood, iliac lymph nodes, liver and spleen did not appear to change upon antigenic challenge, we observed a marked increase in the inguinal lymph nodes of the immunized mice (FIG. 14D). This indicates that some of the T_{H}::iFoxp3 cells expanded in the draining lymph nodes (s.c. immunization into the flanks). However, the number of ‘endogenous’ cells in the inguinal lymph nodes increased equally (FIG. 14E), suggesting that both populations expand to a similar degree with their ratio remaining constant.

### Switching Off Immune Responses

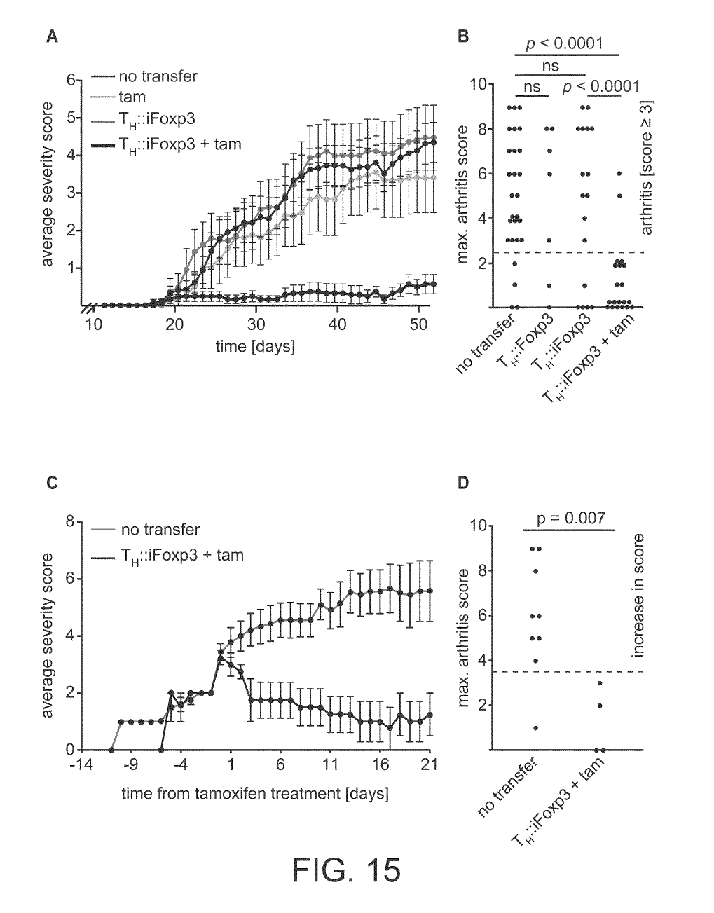

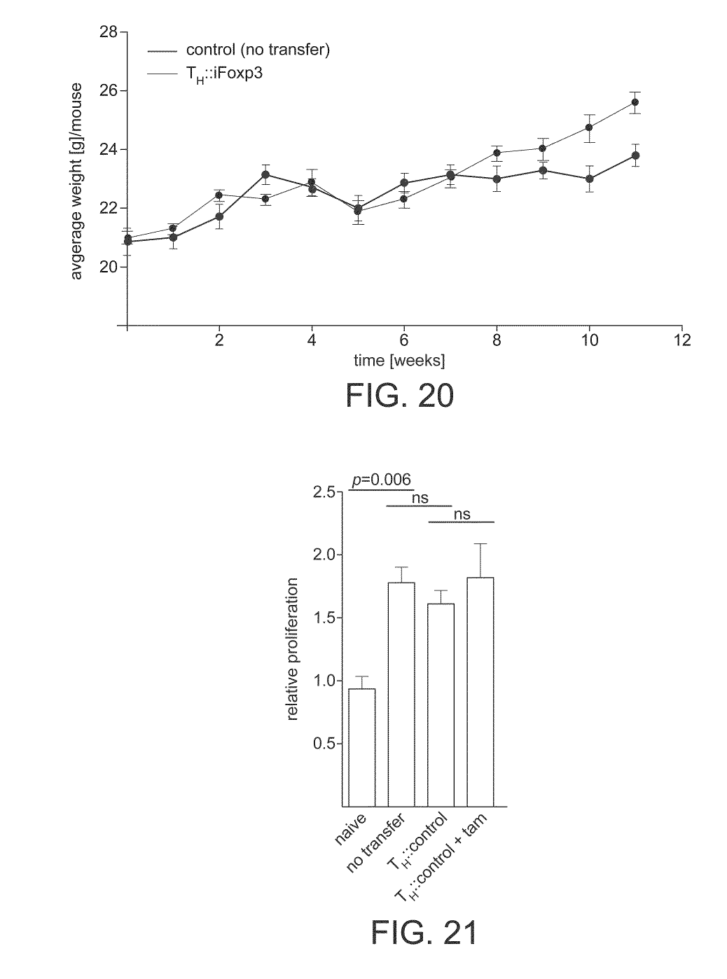

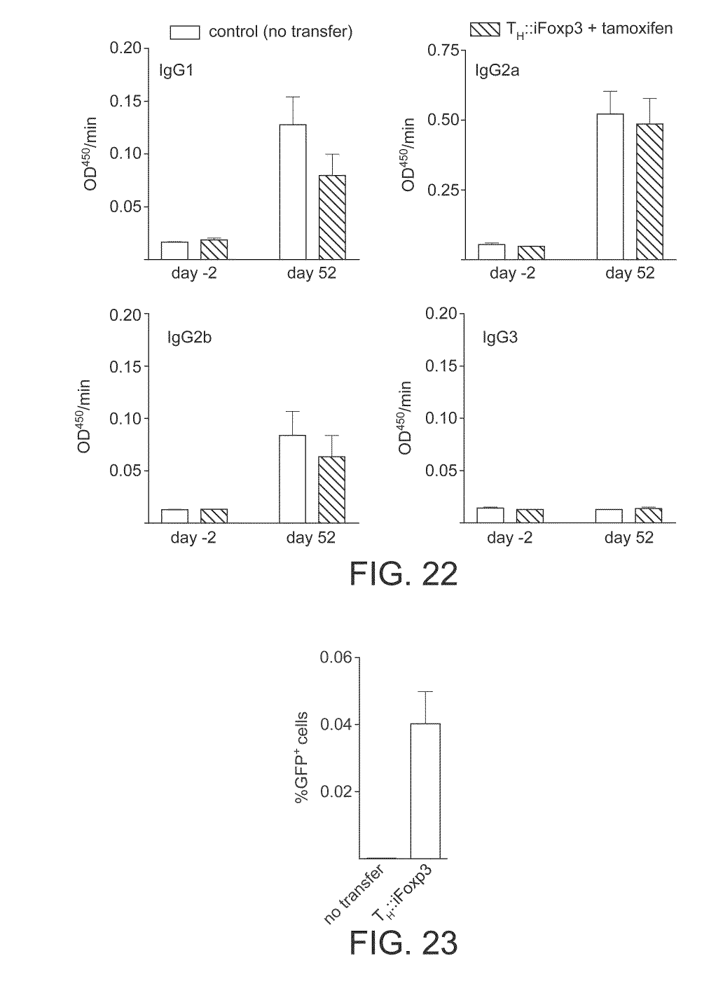

To test the potential of T_{H}::iFoxp3 cells in suppressing autoimmune responses, we turned to the collagen-induced arthritis model, in which T_{H}::Foxp3 cells had failed to show an effect (FIG. 11A). We transferred 1-2×10⁶ polyclonal T_{H}::iFoxp3 cells into wild type DBA/1 mice one day prior to immunization with cII in CFA. We induced iFoxp3 on day 15 after immunization, which lies between the peak of the T cell response to collagen around day 10 [44,45] and the onset of clinical symptoms around day 21 [46]. Mice that had received T_{H}::iFoxp3 cells but did not receive tamoxifen injections to induce iFoxp3 showed the first signs of arthritis on day 19, similar to the mice that received no transfer of cells (FIG. 15A). This effect was specific to the antigenic challenge (cII in CFA) inducing the autoimmune response, as mice receiving these cells without immunization did not exhibit any overt signs of developing autoimmune disease (FIG. 20). Remarkably, 23 out of 25 of the mice that had received T_{H}::iFoxp3 cells and tamoxifen injections to induce iFoxp3 did not show any clinical signs of arthritis (scores <3; FIGS. 15B). This is in stark contrast to the other groups, in which the majority of animals developed arthritis (scores ≧3; FIGS. 15B). Whilst tamoxifen has been reported to have anti-inflammatory properties [47], we found that by itself it had only a minor effect on the development of CIA (FIG. 15A) and no effect on the activity of T_{H}::control cells in vivo (FIG. 21). Despite the clear suppression of the clinical signs of CIA, we could detect collagen-specific antibodies in the serum of the animals at day 52, irrespective of the treatment they had received (FIG. 22).

Next, we investigated whether T_{H}::iFoxp3 cells are capable of stopping already established CIA. To this end, we waited until the mice had reached a clinical score of 3 before inducing iFoxp3. The induction appeared to completely halt if not reverse CIA, leading to a decline in the average severity score (FIG. 15C). None of the mice showed a further increase of symptoms after induction of iFoxp3 (FIG. 15D).

### Specificity of the Suppression

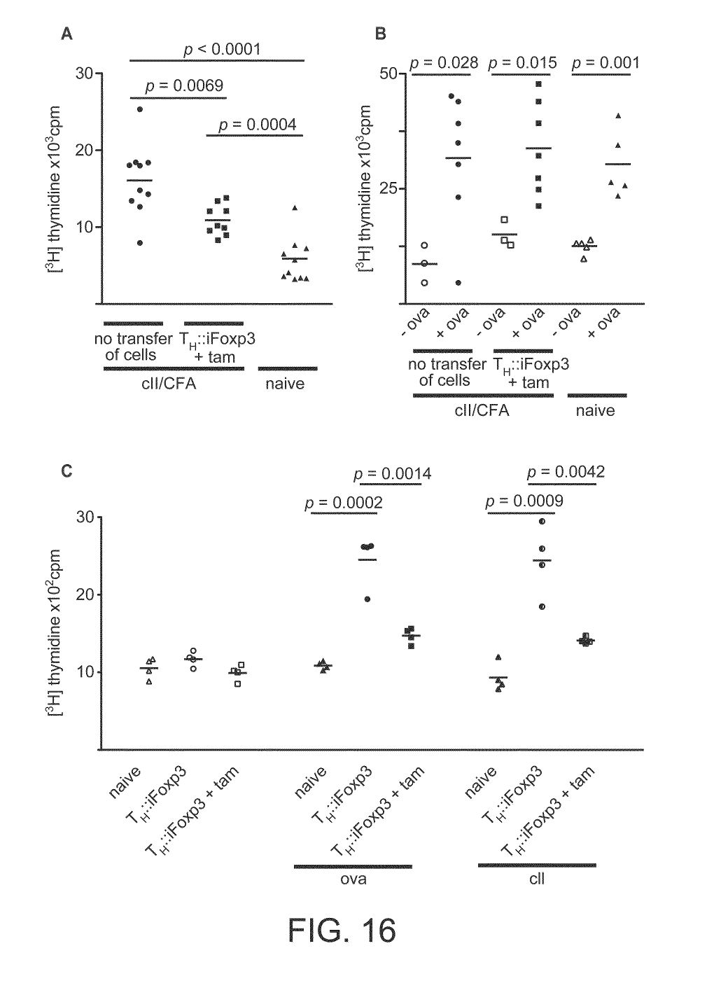

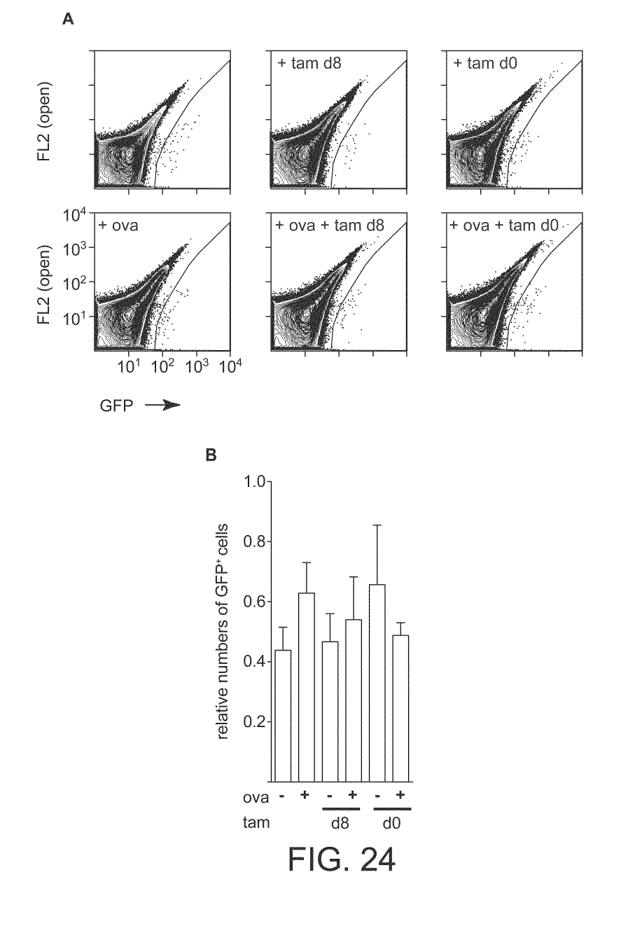

To assess whether the conversion of T_{H}::iFoxp3 cells to T_{R} cell phenotype causes systemic immunosuppression, we compared ‘ex vivo recall reactions’ to the antigen used prior to the induction of iFoxp3 (cII) to that of an unrelated antigen (ova) injected after induction. The collagen-specific T cell proliferation measured for mice in which iFoxp3 had been induced was significantly lower than that of mice that had received no transfer of cells, albeit still higher than that of naive mice (FIG. 16A). As we did not add tamoxifen to the ex vivo culture, this most likely reflects a lower number of cII-specific pro-inflammatory T cells in the animals that had received T_{H}::iFoxp3 cells and tamoxifen induction, rather than a mere ex vivo suppressive effect of T_{H}::iFoxp3 cells. Remarkably, we could not detect any difference in the T cell proliferation upon exposure to ova irrespective of whether the mice had received treatment or not (FIG. 16B). This suggests that the suppression only affects immune responses in which the T_{H}::iFoxp3 cells have had the opportunity to participate prior to induction of iFoxp3. Indeed, we were able to detect T_{H}::iFoxp3 cells in the inflamed paw of cII-immunized mice, suggesting that in the absence of induction these cells can contribute to the inflammation (FIG. 23) However, once converted the T_{H}::iFoxp3 cells, despite still being present (FIGS. 24A and B), seem to have lost the capacity to suppress further unrelated immunological challenges (FIG. 16B). This suggests that the conversion of T_{H}::iFoxp3 cells by induction of iFoxp3 does not lead to a systemic immunosuppression.

Having shown that induced T_{H}::iFoxp3 cells do not suppress further unrelated immune responses post induction, we wanted to investigate the suppressive activity of T_{H}::iFoxp3 cells in a context in which both cII and ova are present prior to induction. We transferred 1×10⁶ polyclonal T_{H}::iFoxp3 cells into wild type DBA/1 mice one day before immunization with a 1:1 mixture of ova and cII in CFA. We induced iFoxp3 on day 15 after immunization and assessed the antigen-induced proliferation of splenocytes prepared from these mice on day 28. The recall proliferation against ova and cII were comparable. Equally similar was the reduction in proliferation in the cases in which iFoxp3 was induced (FIG. 16C). In combination, these results suggest that this approach enables selective suppression without affecting further unrelated immune responses after induction of iFoxp3.

T_{H}::iFoxp3 Cell Longevity

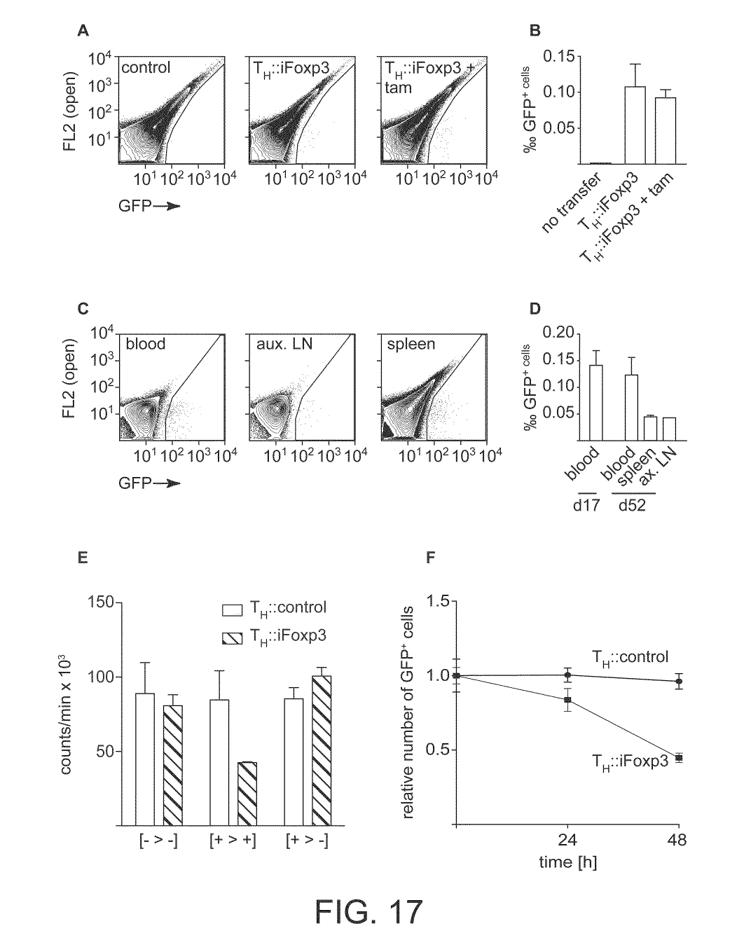

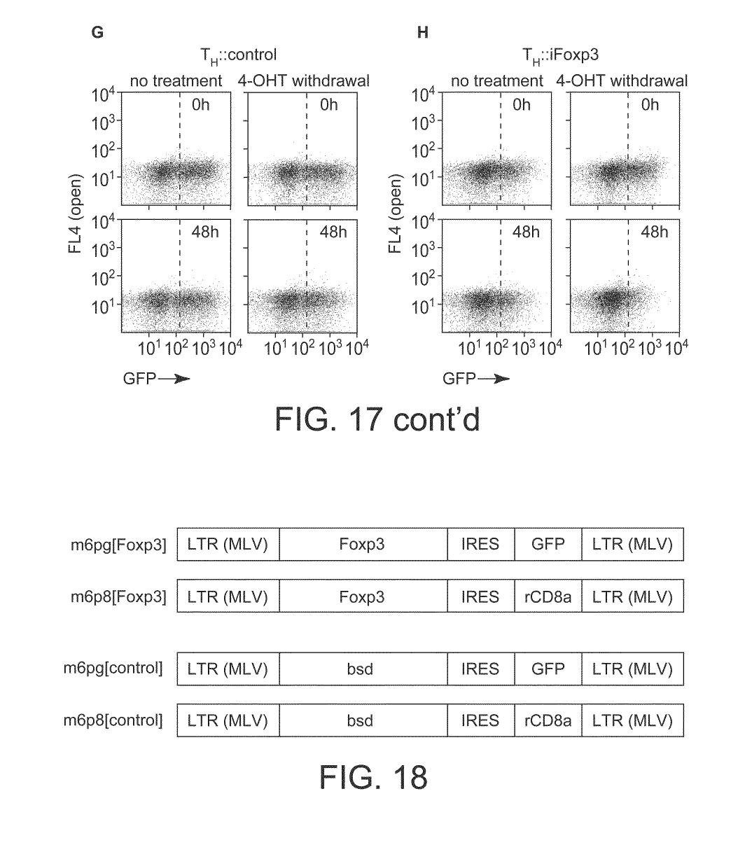

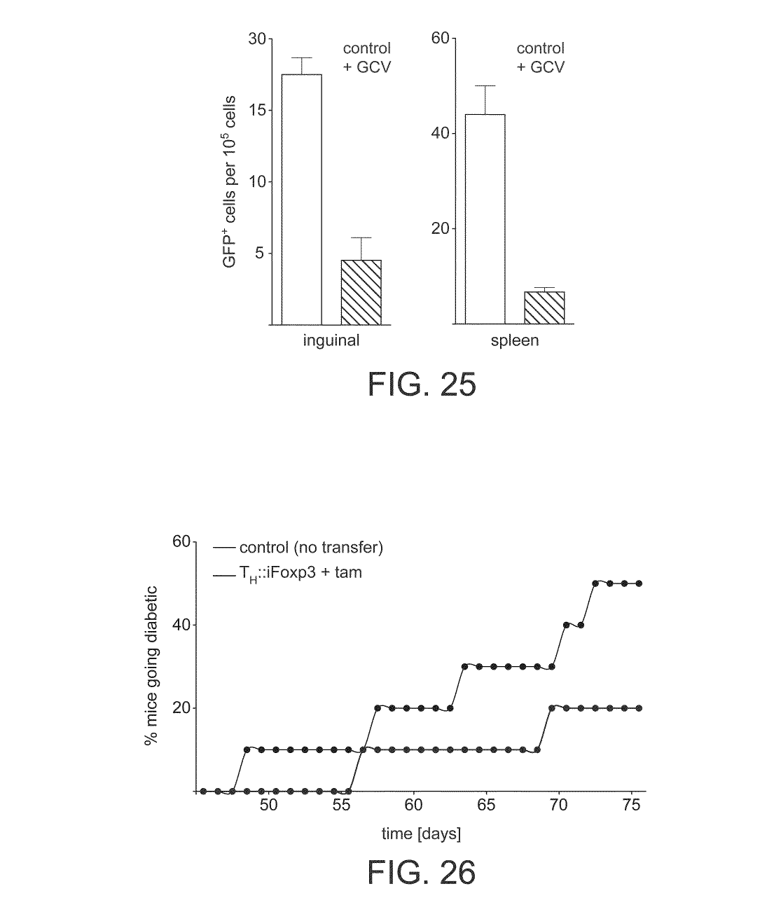

It is noteworthy, that we were able to detect T_{H}::iFoxp3 cells 52 days after their transfer, independent of the level of arthritis and whether the mice had received tamoxifen treatment or not (FIGS. 17A and B). An analysis of various tissues revealed that T_{H}::iFoxp3 cells in blood were only marginally reduced between day 17 and day 52 (FIGS. 17C and D) and could readily be detected in the auxiliary lymph nodes and spleen. Whilst this is likely to be of advantage with regard to actively suppressing immune responses, it poses the question whether continuous tamoxifen presence is required. Due to the long half-life of tamoxifen [48], a direct assessment of this in vivo is not feasible. However, in vitro suppression assays, T_{H}::iFoxp3 cells had completely lost their suppressive activity 72 h after withdrawal of 4-OHT (FIG. 17E). To perform these experiments we had to compensate for a marked reduction in the number of viable T_{H}::iFoxp3 cells that could be recovered under these conditions. To formally address the effect of the withdrawal of 4-OHT on T_{H}::iFoxp3 cell viability, we exposed the cells to 4-OHT for 48 h from the point of transduction and then cultured them for a further 48 h in the absence of 4-OHT. The number of viable cells was assessed by flow cytometry. Withdrawal of 4-OHT had no effect on T_{H}::control cells, but led to a marked decrease in the number of T_{H}::iFoxp3 cells (FIG. 17F to H). This suggests, that once induced, T_{H}::iFoxp3 cells die upon tamoxifen withdrawal, but it remains unclear how this translates into an in vivo context. Indeed, it might be desirable to incorporate a suicide gene [49] into the retroviral vector as this allows the removal of the transduced cells if desired (FIG. 25).

### Discussion

Here, we have demonstrated an approach, which allows us to stop undesirable immune responses without prior knowledge of the antigens involved. T_{H}::iFoxp3 cells participate in immune responses as conventional T_{H} cells until iFoxp3 is induced. At this point they change their phenotype from that of pro-inflammatory T cells to that of regulatory T cells and suppress the response they partook in.

Ectopic expression of Foxp3 in conventional T cells leads to their conversion into cells with T_{R}-like phenotype [19-21]. It was demonstrated early on, that these T_{H}::Foxp3 cells, like T_{R} cells, could suppress the development of colitis in lymphopenic hosts [19,29]. However, it was noted that in this context the effectiveness of both polyclonal T_{H}::Foxp3 cells and T_{R} cells [29,50,51] might be due to the regulation of homeostatic expansion of the co-transferred, pro-inflammatory cells, rather than to a true antigen-specific suppression [9,11,52]. Furthermore, adoptive transfer of polyclonal T_{R} cells will only marginally increase the number of suppressive cells that recognize a particular antigen. Indeed, the use of polyclonal T_{R} cell [22] or T_{H}::Foxp3 populations [11,23] (FIG. 11A) have been of limited efficacy, unless the immune pathology was caused by an absence of functional T_{R} cells [20,53] or the experiments were performed in lymphopenic animals [11]. The restrictions imposed by the low frequency of antigen-specific T_{R} or T_{H}::Foxp3 cells in polyclonal populations can be circumvented by ex vivo expansion of antigen-specific T_{R} cells and TCR transgenic T_{H}::Foxp3 cells [9-11,41]. Both approaches have been successfully exploited in mouse models to treat diabetes [23,24,54,55], arthritis [31] and EAE [56], as well as being used for the induction of transplantation tolerance [57,58]. Whilst TCR transgenic T cells are an invaluable research tool to improve our understanding of the regulation of immune responses [59,60], it is unclear to what degree they can be used in a therapeutic context. Ex vivo expansion of antigen-specific T_{R} cells [9,11], or in vivo conversion of T_{H} into T_{R} cells [12], promises to be more applicable. However, these approaches are technically challenging, time consuming and most importantly require knowledge of or access to the antigens involved in the immune response to be suppressed [8,13].

Our study of T_{H}::Foxp3 cells revealed a further problem. Whilst T_{H}::Foxp3 cells appear to adopt the characteristics of T_{R} cells in vitro, we found their homing to be altered from that of endogenous T_{R} and T_{H} cells. This hinders the T_{H}::Foxp3 cells from mimicking the homing behavior of endogenous T_{R} cells, which has been shown to be important for their suppressive function in vivo [61-63]. Those T_{H}::Foxp3 cells that fail to home to the secondary lymphoid organs might not receive the required antigen priming [63] and thus fail to expand like endogenous T_{R} cells [64]. This might explain the difference in the efficacy of approaches that use polyclonal Foxp3⁺ cells and those that use antigen-selected or TCR transgenic Foxp3⁺ cells. The latter might circumvent the need for an antigen-specific expansion in vivo by ensuring that there are sufficient numbers of antigen-specific cells from the onset.

The activation-induced, Foxp3-mediated down-regulation of CD62L might well be a key factor in the exclusion of T_{H}::Foxp3 cells from the peripheral lymph nodes since T cells from CD62L-deficient mice exhibit a similar phenotype [34,35]. Further, it has been shown that CD62^{hi} polyclonal T_{R} cells have a more potent protective effect in vivo [65]. However, we cannot exclude that ectopic expression of Foxp3 also alters the expression of other homing receptors. Indeed, we found that the activation-induced down-regulation of CD62L in thymically derived T_{R} and T_{H} cells was not sufficient to exclude them from the peripheral lymph nodes.

Here, we present an approach that addresses these problems by transducing polyclonal, conventional T cells with a retroviral vector encoding a genetically engineered inducible form of Foxp3. T_{H}::iFoxp3 cells retain their pro-inflammatory character and the ability to home to the lymph nodes. Those T_{H}::iFoxp3 cells that recognize an antigen appear to participate in the immune response and expand. This in vivo expansion of antigen-specific T_{H}::iFoxp3 cells circumvents the need for an ex vivo expansion and does not rely on any knowledge of the antigens involved. Upon induction of iFoxp3, the in vivo expanded, antigen-specific T_{H}::iFoxp3 cells assume a T_{R} cell-like phenotype and suppress the undesirable response they initially partook in. We were able to demonstrate the efficacy of our approach by specifically halting collagen-induced arthritis in a mouse model. Importantly, T_{H}::iFoxp3 cell-mediated suppression appears to be restricted to the specific response, which is ongoing at the time of induction of iFoxp3. Those T_{H}::iFoxp3 cells that do not already participate in an immune response at the time of induction lose the capacity to suppress further unrelated immune responses despite still being present. Whilst we cannot exclude that other factors play a role, it appears that the antigen specific expansion of the T_{H}::iFoxp3 cells prior to induction is an integral part of the observed non-systemic suppression. In a therapeutic context, it might be desirable to limit the exposure to tamoxifen to minimize possible side effects. Whilst it appears that most T_{H}::iFoxp3 cells die upon withdrawal of tamoxifen, those that do survive lose their suppressive activity. To avoid possible deleterious effects these ‘revertant’ cells can be removed based on the incorporation of a suicide gene into the retroviral vector used for the delivery of iFoxp3.

We believe that this strategy of induced conversion of T_{H} cells into cells with T_{R} cell-like phenotype using iFoxp3 is generally applicable and allows us to stop a variety of undesirable immune responses.

### REFERENCES TO EXAMPLE 5

1. Rioux J D, Abbas A K (2005) Paths to understanding the genetic basis of autoimmune disease. Nature 435: 584-589.
2. Gummert J F, Ikonen T, Morris R E (1999) Newer Immunosuppressive Drugs: A Review. Journal of the American Society of Nephrology J Am Soc Nephrol 10: 1366-1380.
3. St Clair E W, Turka L A, Saxon A, Matthews J B, Sayegh M H et al. (2007) New reagents on the horizon for immune tolerance. Annu Rev Med 58: 329-346.
4. Sakaguchi S (2004) Naturally arising CD4+ regulatory t cells for immunologic self-tolerance and negative control of immune responses. Annu Rev Immunol 22: 531-562.
5. Aluvihare V R, Kallikourdis M, Betz A G (2004) Regulatory T cells mediate maternal tolerance to the fetus. Nat Immunol 5: 266-271.
6. Trowsdale J, Betz A G (2006) Mother's little helpers: mechanisms of maternal-fetal tolerance. Nat Immunol 7: 241-246.
7. Darrasse-Jeze G, Klatzmann D, Charlotte F, Salomon B L, Cohen J L (2006) CD4+CD25+ regulatory/suppressor T cells prevent allogeneic fetus rejection in mice. Immunol Lett 102: 106-109.
8. Bluestone J A, Thomson A W, Shevach E M, Weiner H L (2007) What does the future hold for cell-based tolerogenic therapy? Nat Rev Immunol 7: 650-654.
9. Bluestone J A (2005) Regulatory T-cell therapy: is it ready for the clinic? Nat Rev Immunol 5: 343-349.
10. von Boehmer H (2006) Can studies of tolerance ever lead to therapy? Ann Rheum Dis 65 Suppl 3: iii41-iii43.
11. Masteller E L, Tang Q, Bluestone J A (2006) Antigen-specific regulatory T cells—ex vivo expansion and therapeutic potential. Semin Immunol 18: 103-110.
12. Kretschmer K, Apostolou I, Hawiger D, Khazaie K, Nussenzweig M C et al. (2005) Inducing and expanding regulatory T cell populations by foreign antigen. Nat Immunol 6: 1219-1227.
13. Kretschmer K, Heng T S, von Boehmer H (2006) De novo production of antigen-specific suppressor cells in vivo. Nat Protoc 1: 653-661.
14. Sakaguchi S (2005) Naturally arising Foxp3-expressing CD25+CD4+ regulatory T cells in immunological tolerance to self and non-self. Nat Immunol 6: 345-352.
15. Zheng Y, Rudensky A Y (2007) Foxp3 in control of the regulatory T cell lineage. Nat Immunol 8: 457-462.
16. Bettelli E, Carrier Y, Gao W, Korn T, Strom T B et al. (2006) Reciprocal developmental pathways for the generation of pathogenic effector TH17 and regulatory T cells. Nature 441: 235-238.
17. Chen W, Jin W, Hardegen N, Lei K J, Li L et al. (2003) Conversion of peripheral CD4+CD25− naive T cells to CD4+CD25+ regulatory T cells by TGF-beta induction of transcription factor Foxp3. J Exp Med 198: 1875-1886.
18. Fantini M C, Becker C, Monteleone G, Pallone F, Galle P R et al. (2004) Cutting edge: TGF-beta induces a regulatory phenotype in CD4+CD25− T cells through Foxp3 induction and down-regulation of Smad7. J Immunol 172: 5149-5153.
19. Hori S, Nomura T, Sakaguchi S (2003) Control of regulatory T cell development by the transcription factor Foxp3. Science 299: 1057-1061.
20. Fontenot J D, Gavin M A, Rudensky A Y (2003) Foxp3 programs the development and function of CD4+CD25+regulatory T cells. Nat Immunol 4: 330-336.
21. Khattri R, Cox T, Yasayko S A, Ramsdell F (2003) An essential role for Scurfin in CD4+CD25+ T regulatory cells. Nat Immunol 4: 337-342.
22. Tarbell K V, Yamazaki S, Olson K, Toy P, Steinman R M (2004) CD25+CD4+ T cells, expanded with dendritic cells presenting a single autoantigenic peptide, suppress autoimmune diabetes. J Exp Med 199: 1467-1477.
23. Jaeckel E, von Boehmer H, Maims MP (2005) Antigen-specific FoxP3-transduced T-cells can control established type 1 diabetes. Diabetes 54: 306-310.
24. Green E A, Choi Y, Flavell R A (2002) Pancreatic lymph node-derived CD4(+)CD25(+) Treg cells: highly potent regulators of diabetes that require TRANCE-RANK signals. Immunity 16: 183-191.
25. Floess S, Freyer J, Siewert C, Baron U, Olek S et al. (2007) Epigenetic control of the foxp3 locus in regulatory T cells. PLoS Biol 5: e38.
26. Selvaraj R K, Geiger T L (2007) A Kinetic and Dynamic Analysis of Foxp3 Induced in T Cells by TGF-beta. J Immunol 178: 7667-7677.
27. Mucida D, Park Y, Kim G, Turovskaya O, Scott I et al. (2007) Reciprocal TH17 and regulatory T cell differentiation mediated by retinoic acid. Science 317: 256-260.
28. Sun C M, Hall J A, Blank R B, Bouladoux N, Oukka M et al. (2007) Small intestine lamina propria dendritic cells promote de novo generation of Foxp3 T reg cells via retinoic acid. J Exp Med 204: 1775-1785.
29. Mottet C, Uhlig H H, Powrie F (2003) Cutting edge: cure of colitis by CD4+CD25+ regulatory T cells. J Immunol 170: 3939-3943.
30. Loser K, Hansen W, Apelt J, Balkow S, Buer J et al. (2005) In vitro-generated regulatory T cells induced by Foxp3-retrovirus infection control murine contact allergy and systemic autoimmunity. Gene Ther 12: 1294-1304.
31. Fujio K, Okamoto A, Araki Y, Shoda H, Tahara H et al. (2006) Gene Therapy of Arthritis with TCR Isolated from the Inflamed Paw. J Immunol 177: 8140-8147.
32. Brand D D, Kong A H, Rosloniec E F (2003) Immunopathogenesis of collagen arthritis. Springer Semin Immunopathol 25: 3-18.
33. Boehm T, Bleul C C (2007) The evolutionary history of lymphoid organs. Nat Immunol 8: 131-135.
34. Arbones M L, Ord D C, Ley K, Ratech H, Maynard-Curry C et al. (1994) Lymphocyte homing and leukocyte rolling and migration are impaired in L-selectin-deficient mice. Immunity 1: 247-260.
35. Rosen S D (2004) Ligands for L-selectin: homing, inflammation, and beyond. Annu Rev Immunol 22: 129-156.
36. Smalley D M, Ley K (2005) L-selectin: mechanisms and physiological significance of ectodomain cleavage. J Cell Mol Med 9: 255-266.
37. Wu Y, Borde M, Heissmeyer V, Feuerer M, Lapan A D et al. (2006) FOXP3 controls regulatory T cell function through cooperation with NFAT. Cell 126: 375-387.
38. Ono M, Yaguchi H, Ohkura N, Kitabayashi I, Nagamura Y et al. (2007) Foxp3 controls regulatory T-cell function by interacting with AML1/Runx1. Nature 446: 685-689..
39. Marson A, Kretschmer K, Frampton G M, Jacobsen E S, Polansky J K et al. (2007) Foxp3 occupancy and regulation of key target genes during T-cell stimulation. Nature 445: 931-935.
40. Zheng Y, Josefowicz S Z, Kas A, Chu T T, Gavin M A et al. (2007) Genome-wide analysis of Foxp3 target genes in developing and mature regulatory T cells. Nature 445: 936-940.
41. Roncarolo M G, Battaglia M (2007) Regulatory T-cell immunotherapy for tolerance to self antigens and alloantigens in humans. Nat Rev Immunol 7: 585-598.
42. Feil R, Wagner J, Metzger D, Chambon P (1997) Regulation of Cre recombinase activity by mutated estrogen receptor ligand-binding domains. Biochem Biophys Res Commun 237: 752-757.
43. Rossant J, McMahon A (1999) “Cre”-ating mouse mutants—a meeting review on conditional mouse genetics. Genes and Development Genes Dev 13: 142-145.
44. Pogue-Caley R R, McHeyzer-Williams M G (2001) Emergence of a type II collagen-specific helper T cell response. Eur J Immunol 31: 2362-2372.
45. Latham K A, Whittington K B, Zhou R, Qian Z, Rosloniec E F (2005) Ex vivo characterization of the autoimmune T cell response in the HLA-DR1 mouse model of collagen-induced arthritis reveals long-term activation of type II collagen-specific cells and their presence in arthritic joints. J Immunol 174: 3978-3985.
46. Rosloniec E F, Cremer M, Kang A, Myers L K (2001) Collagen-induced Arthritis. Current Protocols in Immunology 15.5: 15.5.1-15.5.24.
47. Grainger D J, Metcalfe J C (1996) Tamoxifen: teaching an old drug new tricks? Nat Med 2: 381-385.
48. Guerrieri-Gonzaga A, Baglietto L, Johansson H, Bonanni B, Robertson C et, al. (2001) Correlation between tamoxifen elimination and biomarker recovery in a primary prevention trial. Cancer Epidemiol Biomarkers Prey 10: 967-970.
49. Straathof K C, Spencer D M, Sutton R E, Rooney C M (2003) Suicide genes as safety switches in T lymphocytes. Cytotherapy 5: 227-230.
50. Read S, Malmstrom V, Powrie F (2000) Cytotoxic T lymphocyte-associated antigen 4 plays an essential role in the function of CD25(+)CD4(+) regulatory cells that control intestinal inflammation. J Exp Med 192: 295-302.
51. Morgan M E, Bierman R, van Duivenvoorde L M, Witteveen H J, van Ewijk W et al. (2005) Effective treatment of collagen-induced arthritis by adoptive transfer of CD25+ regulatory T cells. Arthritis Rheum 52: 2212-2221.
52. Li J, Bracht M, Shang X, Radewonuk J, Emmen E et al. (2006) Ex vivo activated OVA specific and non-specific CD4+CD25+ regulatory T cells exhibit comparable suppression to OVA mediated T cell responses. Cell Immunol 241: 75-84.
53. Asano M, Toda M, Sakaguchi N, Sakaguchi S (1996) Autoimmune disease as a consequence of developmental abnormality of a T cell subpopulation. J Exp Med 184: 387-396.
54. Tang Q, Henriksen K J, Bi M, Finger E B, Szot G et al. (2004) In vitro-expanded antigen-specific regulatory T cells suppress autoimmune diabetes. J Exp Med 199: 1455-1465.
55. Tarbell K V, Petit L, Zuo X, Toy P, Luo X et al. (2007) Dendritic cell-expanded, islet-specific CD4+CD25+CD62L+ regulatory T cells restore normoglycemia in diabetic NOD mice. J Exp Med 204: 191-201.
56. Hori S, Haury M, Coutinho A, Demengeot J (2002) Specificity requirements for selection and effector functions of CD25+4+ regulatory T cells in anti-myelin basic protein T cell receptor transgenic mice. Proc Natl Acad Sci USA 99: 8213-8218.
57. Chai J G, Xue S A, Coe D, Addey C, Bartok I et al. (2005) Regulatory T cells, derived from naive CD4+CD25− T cells by in vitro Foxp3 gene transfer, can induce transplantation tolerance. Transplantation 79: 1310-1316.
58. Battaglia M, Stabilini A, Roncarolo M G (2005) Rapamycin selectively expands CD4+CD25+ FoxP3+ regulatory T cells. Blood 105: 4743-4748.

59. Bluthmann H, Kisielow P, Uematsu Y, Malissen M, Krimpenfort P et al. (1988) T-cell-specific deletion of T-cell receptor transgenes allows functional rearrangement of endogenous alpha- and beta-genes. Nature 334: 156-159.
60. Stefanova I, Dorfman J R, Germain R N (2002) Self-recognition promotes the foreign antigen sensitivity of naive T lymphocytes. Nature 420: 429-434.
61. Nguyen V H, Zeiser R, Dasilva D L, Chang D S, Beilhack A et al. (2006) In vivo dynamics of regulatory T cell trafficking and survival predict effective strategies to control graft-versus-host disease following allogeneic transplantation. Blood 109: 2649-2656.
62. Sather B D, Treuting P, Perdue N, Miazgowicz M, Fontenot J D et al. (2007) Altering the distribution of Foxp3+ regulatory T cells results in tissue-specific inflammatory disease. J Exp Med 204: 1335-47.
63. Ochando J C, Yopp A C, Yang Y, Garin A, Li Y et al. (2005) Lymph node occupancy is required for the peripheral development of alloantigen-specific Foxp3+ regulatory T cells. J Immunol 174: 6993-7005.
64. Klein L, Khazaie K, von Boehmer H (2003) In vivo dynamics of antigen-specific regulatory T cells not predicted from behavior in vitro. Proc Natl Acad Sci USA 100: 8886-8891.
65. Taylor P A, Panoskaltsis-Mortari A, Swedin J M, Lucas P J, Gress R E et al. (2004) L-Selectin(hi) but not the L-selectin(lo) CD4+25+ T-regulatory cells are potent inhibitors of GVHD and BM graft rejection. Blood 104: 3804-3812.
66. Kallikourdis M, Andersen K G, Welch K A, Betz A G (2006) Alloantigen-enhanced accumulation of CCR5+ ‘effector’ regulatory T cells in the gravid uterus. Proc Natl Acad Sci USA 104: 594-599.
67. Fontenot J D, Rasmussen J P, Williams L M, Dooley J L, Farr A G et al. (2005) Regulatory T cell lineage specification by the forkhead transcription factor foxp3. Immunity 22: 329-341.

### Example 6

### Application in Diabetes

TH::iFoxp3 cells can suppress the development of diabetes.

Diabetes was induced on day 0 by transferring 15×10⁶ unfractionated splenocytes from NOD donors into NODxSCID recipients.

With reference to FIG. 26, the treatment group received 1×10⁶ TH::iFoxp3 cells (red (grey), n=10) and tamoxifen injections. The control group did not receive any further treatment (black (black), n=10).

Thus it can be clearly seen that the number of mice going diabetic continues to climb, and climbs more steeply, in the control group. By contrast, in the group of mice treated according to the invention which received T cells comprising inducible lineage factor (in this example iFoxp3) and in which the lineage factor activity was induced (in this example by administration of tamoxifen), fewer mice went diabetic, and of those mice which did go diabetic, onset was delayed.

Thus the applicability of the invention to treatment or prevention of diabetes is demonstrated.

### Example 7

### Phenotype Switching (Th₀/Th17)

In this example we further demonstrate phenotype switching according to the present invention. In this example the switching is done in vitro.

In this example, the inducible lineage factor is RORgt. The induction is via addition of tamoxifen (the RORgt is provided as an ERT fusion).

By intracellular staining we looked at the expression levels of the key signature cytokines INFg (expressed by TH1. cells), IL4 (expressed by the TH2 cells) and IL17 (expressed by TH17 cells) in iRORgt, RORgt or MOCK transduced cells grown in cultures with or without tamoxifen.

In iRORgt transduced cells grown without tamoxifen we detect no IL17 expression as is the case for MOCK transduced cells. When the iRORgt cells have been grown with tamoxifen we clearly see an increased IL17 expression which is similar to the IL17 expression we observe in cells transduced with the constitutively active RORgt construct. As expected we see no significant change in the expression levels of INFg or IL4 in iRORgt or RORgt transduced cells.

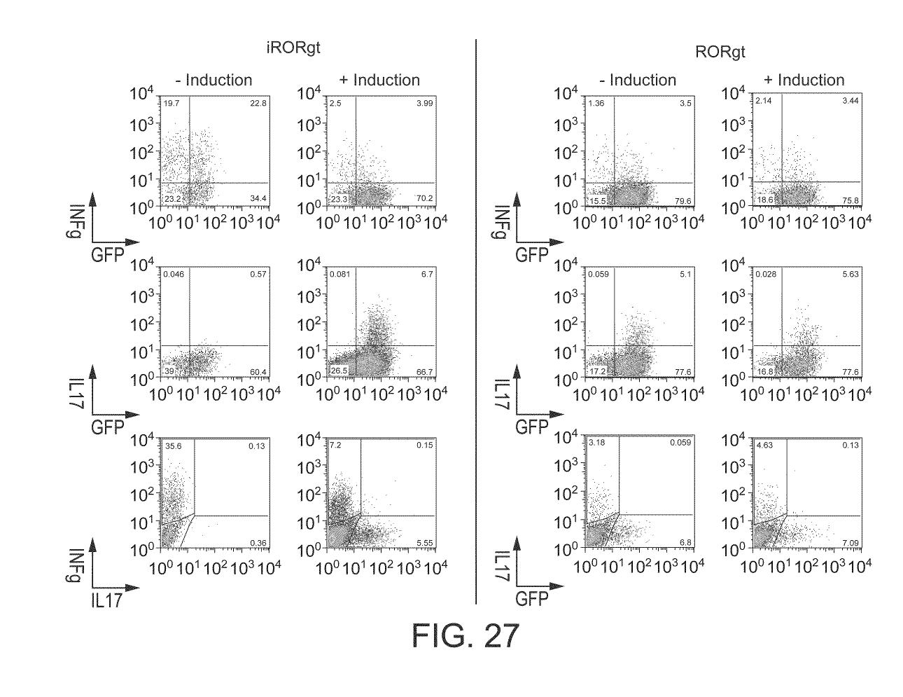

The results are shown in FIG. 27. The plots shown are gated on lymphocytes and the numbers in the quadrants indicate the percentage of total lymphocytes. RCD8 and GFP indicates transduction efficiency.

This demonstrates the capability of turning naive T cells into IL17 expressing T cells according to the invention (Th₀-Th17 switching). Moreover, it is shown that this is done in a controlled and inducible way.

### Example 8

### Phenotype Switching (Th1/Th17)

Further to example 7, in this example we looked at the effect of iRORgt induction in cells that have been grown in TH1. polarizing conditions (grown in cultures with 20 ng/mL IL12).

The clear effect of the induction of iRORgt is an increased expression of IL17. This indicates that the induction of iRORgt according to the invention is sufficient to switch cells into TH17 cells even though the cytokine environment favours TH1 polarization. Furthermore, we observe a significant decrease in the percentage of INFg expressing cells in cultures where iRORgt has been induced. This seems to be the case both for transduced and non transduced cells.

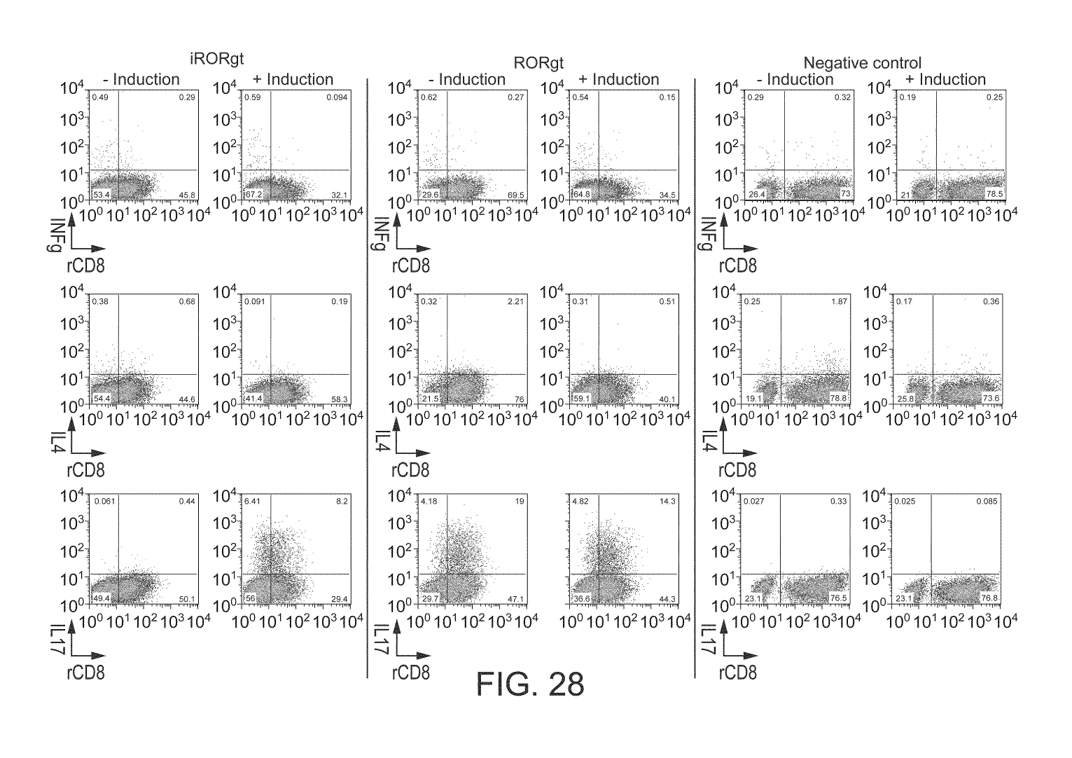

The results are shown in FIG. 28. The plots shown are gated on lymphocytes and the numbers in the quadrants indicate the percentage of total lymphocytes, GFP indicates transduction efficiency. The negative control was very similar to the iRORgt induction and has not been included here.

Again we see that the effects of the induction of iRORgt are very similar to the effects of the constitutively active RORgt, confirming that the inducible construct is fully functional.

Moreover, we see that practically all IL17 expressing cells do not express INFg and vice versa, indicating that the result of iRORgt induction is a complete switch to TH17 cells and not to some TH1/TH17 hybrid.

Thus Th1-Th17 switching according to the invention is demonstrated.

### REFERENCES

1. Sakaguchi, S. Naturally arising CD4+ regulatory t cells for immunologic self-tolerance and negative control of immune responses. Annu Rev Immunol 22, 531-562 (2004).
2. Aluvihare, V. R., Kallikourdis, M. & Betz, A. G. Regulatory T cells mediate maternal tolerance to the fetus. Nat Immunol 5, 266-271 (2004).
3. Hori, S., Nomura, T. & Sakaguchi, S. Control of regulatory T cell development by the transcription factor Foxp3. Science 299, 1057-1061 (2003).
4. Fontenot, J. D., Gavin, M. A. & Rudensky, A. Y. Foxp3 programs the development and function of CD4+CD25+regulatory T cells. Nat Immunol 4, 330-336 (2003).
5. Khattri, R., Cox, T., Yasayko, S. A. & Ramsdell, F. An essential role for Scurfin in CD4+CD25+T regulatory cells. Nat Immunol 4, 337-342 (2003).
6. Zheng, Y. & Rudensky, A. Y. Foxp3 in control of the regulatory T cell lineage. Nat Immunol 8, 457-462 (2007).
7. Jaeckel, E., von Boehmer, H. & Maims, M. P. Antigen-specific FoxP3-transduced T-cells can control established type 1 diabetes. Diabetes 54, 306-310 (2005).
8. Fujio, K. et al. Gene Therapy of Arthritis with TCR Isolated from the Inflamed Paw. J Immunol 177, 8140-8147 (2006).
9. Wang, R. et al. Foxp3-expressing CD4(+)T Cells Under the Control of IFN-gamma Promoter Prevent Diabetes in NOD Mice. Mol Ther (2007).
10. von Boehmer, H. Can studies of tolerance ever lead to therapy? Ann Rheum Dis 65 Suppl 3, iii41-iii43 (2006).
11. Bluestone, J. A. Regulatory T-cell therapy: is it ready for the clinic? Nat Rev Immunol 5, 343-349 (2005).

12. Loser, K. et al. In vitro-generated regulatory T cells induced by Foxp3-retrovirus infection control murine contact allergy and systemic autoimmunity. Gene Ther (2005).
13. Kallikourdis, M., Andersen, K. G., Welch, K. A. & Betz, A. G. Alloantigen-enhanced accumulation of CCR5+ ‘effector’ regulatory T cells in the gravid uterus. Proc Natl Acad Sci USA 104, 594-599 (2006).
14. Gallatin, W. M., Weissman, I. L. & Butcher, E. C. A cell-surface molecule involved in organ-specific homing of lymphocytes. Nature 304, 30-34 (1983).
15. Taylor, P. A. et al. L-Selectin(hi) but not the L-selectin(lo) CD4+25+ T-regulatory cells are potent inhibitors of GVHD and BM graft rejection. Blood 104, 3804-3812 (2004).
16. Rosen, S. D. Ligands for L-selectin: homing, inflammation, and beyond. Annu Rev Immunol 22, 129-156 (2004).
17. Klein, L., Khazaie, K. & von Boehmer, H. In vivo dynamics of antigen-specific regulatory T cells not predicted from behavior in vitro. Proc Natl Acad Sci USA 100, 8886-8891 (2003).
18. Feil, R., Wagner, J., Metzger, D. & Chambon, P. Regulation of Cre recombinase activity by mutated estrogen receptor ligand-binding domains. Biochem Biophys Res Commun 237, 752-757 (1997).
19. Rosloniec, E. F., Cremer, M., Kang, A. & Myers, L. K. Collagen-induced Arthritis. Current Protocols in Immunology 15.5, (2001).
20. Grainger, D. J. & Metcalfe, J. C. Tamoxifen: teaching an old drug new tricks? Nat Med 2, 381-385 (1996).
21. Tang, Q. et al. In vitro-expanded antigen-specific regulatory T cells suppress autoimmune diabetes. J Exp Med 199, 1455-1465 (2004).
22. Nishimura, E., Sakihama, T., Setoguchi, R., Tanaka, K. & Sakaguchi, S. Induction of antigen-specific immunologic tolerance by in vivo and in vitro antigen-specific expansion of naturally arising Foxp3+CD25+CD4+ regulatory T cells. Int Immunol 16, 1189-1201 (2004).
23. Tarbell, K. V., Yamazaki, S., Olson, K., Toy, P. & Steinman, R. M. CD25+CD4+ T cells, expanded with dendritic cells presenting a single autoantigenic peptide, suppress autoimmune diabetes. J Exp Med 199, 1467-1477 (2004).
24. Porteus, M. H., Connelly, J. P. & Pruett, S. M. A look to future directions in gene therapy research for monogenic diseases. PLoS Genet 2, e133 (2006).
25. Straathof, K. C., Spencer, D. M., Sutton, R. E. & Rooney, C. M. Suicide genes as safety switches in T lymphocytes. Cytotherapy 5, 227-230 (2003).
26. Fontenot, J. D. et al. Regulatory T cell lineage specification by the forkhead transcription factor foxp3. Immunity 22, 329-341 (2005).
27. Campbell, I. K. et al. Protection from collagen-induced arthritis in granulocyte-macrophage colony-stimulating factor-deficient mice. J Immunol 161, 3639-3644 (1998).

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1-24. (canceled)

25. A method for rendering a population of T-cells susceptible to induced phenotype switching comprising:
transducing one or more cells of a population of T-cells with at least one transgene encoding a fusion protein that comprises at least a portion of one lineage factor, and at least a portion of one control peptide that binds to a selected ligand capable of inducing the lineage factor, such that upon exposure of the transduced cells to a concentration of the ligand effective to bind to the control peptide and induce the lineage factor, phenotype switching of the cells is induced;
wherein the transduction is carried out in vivo or ex vivo and wherein said transduced cells are suitable for introduction into a mammal.

26. The method of claim 25, wherein the T-cells are CD4+ T-cells, and said lineage factor is selected from the group consisting of GATA3, T-bet, RORγt, and Foxp3.

27. The method of claim 25, wherein the control peptide is a modified estrogen receptor.

28. The method according to claim 25, wherein said one transgene encoding a fusion protein that comprises at least a portion of one lineage factor comprises the Foxp3 polypeptide encoded within SEQ ID NO:3.

29. The method of claim 25, wherein the T-cells are CD8+ T-cells, and said lineage factor is eomesodermin.

30. The method according to claim 25, wherein said T-cells are T-helper cells, and the phenotype is switched to a regulatory T cell phenotype following induction of lineage factor activity.

31. A method for suppressing an immune response in a mammal, comprising introducing into the mammal the population of T-cells of claim 25.

32. A method for rendering a population of T-cells susceptible to induced phenotype switching from T-helper cells to T-reg cells comprising:
transducing one or more cells of a population of T-helper cells with at least one transgene encoding a fusion protein that comprises at least a portion of Foxp3 lineage factor and at least a portion of one estrogen receptor that binds to tamoxifen, which tamoxifen is capable of inducing the Fox3p lineage factor, such that upon exposure of the transduced T-cells to a concentration of tamoxifen effective to bind to the estrogen receptor and induce the Fox3p lineage factor, phenotype switching of the T-cells from T-helper cells to T-reg cells is induced;
wherein the transduction is carried out in vivo or ex vivo and wherein said transduced cells are suitable for introduction into a mammal.

33. The method of claim 32, wherein the transgene encoding a fusion protein that comprises at least a portion of Foxp3 lineage factor encodes the Foxp3 polypeptide encoded within SEQ ID NO:3.

34. A transduced T-cell capable of induced phenotype switching comprising:
at least one transgene encoding a fusion protein that comprises at least a portion of one lineage factor and at least a portion of one control peptide that binds to a selected ligand capable of inducing the lineage factor, such that upon exposure of the transduced T-cell to a concentration of the ligand effective to bind to the control peptide and induce the lineage factor, phenotype switching of the cell is induced;
wherein said transduced T-cell is suitable for introduction into a mammal.

35. The transduced T-cell of claim 34, wherein said lineage factor is further fused to a nucleotide sequence encoding a fluorescent protein.

36. The transduced T-cell of claim 34, wherein the lineage factor is selected from the group consisting of GATA3, T-bet, Eomesodermin, RORγt, and Foxp3.

37. The transduced T-cell of claim 36, wherein said lineage factor comprises the Foxp3 polypeptide encoded within SEQ ID NO:3.

38. The transduced T-cell of claim 34, wherein the control peptide is a modified estrogen receptor.


SEQUENCE LISTING (13 sequences)

SEQ ID NO: 1 - DNA, 2262 bp, Artificial
  nucleic acid construct/primer sequence
atgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgccctgtgccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttga

SEQ ID NO: 2 - protein, 753 aa, Artificial
  polypeptide sequence
MPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCPVPADDTYRYISAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 3 - DNA, 8312 bp, Artificial
  nucleic acid construct/primer sequence
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactaggactagtcatctttttttaagctcaagttttgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcggggtctttcatttggaggttccaccgagatttggagacccctgcccagggaccaccgacccccccgccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgaagctatagaagcttctcccggcaacttctcctgactctgccttcagacgagacttggaagacagtcacatctcagcagctcctctgccgttatccagcctgcctctgacaagaacccaatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgccctgtgccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatgaagcggccgcccctctccctcccccccccctaacgttactggccgaagccgcttggaataaggccggtgtgcgtttgtctatatgttattttccaccatattgccgtcttttggcaatgtgagggcccggaaacctggccctatcttcttgatgagcattcctaggggtctttcccctctcgccaaaggaatgcaaggtctgttgaatgtcgtgaaggaagcagttcctctggaagtttcttgaagataaacaacgtctgtagcaaccctttgcaggcagcggaaccccccacctggcgacaggtgcctctgcggccaaaagccacgtgtataagatacacctgtaaaggcggcacaaccccagtgccacgttgtgagttgggtagttgtggaaagagtcaaatggctctcctcaagcgtattcaacaaggggctgaaggatgcccagaaggtaccccattgtatgggatctgatctggggcctcggtgcatatgctttacatatgtttagtcgaggttaaaaaacgtctaggccccccgaaccacggggacgtggttttcctttgaaaaacacgatgataatatggccacaaccatgcagcttgccagcatgggctacctgcgccgcatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcgccgtaggcaggtagttaacagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcggggtctttcagtttctcccacctacacaggtctcactggatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 4 - DNA, 3117 bp, Artificial
  nucleic acid construct/primer sequence
atgcagttgggagagcagctcctggtgagctcggtgaacctgcccggcgcgcacttctactcgctggagagtgctcgcggaggaggaggaggaggaggcggaggaggagggggaggcggagggagcgtcagcctcctccccggtgctgccccctcgccccagaggctggacttagacaaagcgtccaagaagtttccgggcagtctcccgtgccaggcggggagcgcagaacccgcaggcgctggcgcgggggcccccgcggccatgctcagtgacgcggacgctggggacaccttcgggagcacctcggcggtggccaagcccgggcccccggacggccgcaagggctccccgtgcgcggaggaggagctgccctccgccgccaccgccgcggccaccgcgcgctactccatggacagcctgagctccgagcgctactacctcccgtcgccgggaccgcagggctccgagctcgccgcgccctgctcgctcttccagtacccggcggcggccggagcagcccacggacccgtgtaccccgcgtccaatggcgcgcgctacccctacggctccatgctgccccccggtggattccccgccgccgtgtgcccgcccgcgagggcgcagttcggccccgctgcgggttcggggagcggcgctggtagcagcggcggtggtgctggcggtcctggcgcctatccctacggccagggttctccgctctacgggccatacgccggaacctcagcggccgggtcttgtggaggattggggggcctaggggtgcctggctccggcttccgcgcccacgtctacctgtgcaaccggcccctatggctcaaattccaccggcaccaaactgagatgatcatcaccaaacagggcaggcgcatgtttcctttcttgagcttcaacataaacggactcaaccccaccgcccactacaatgttttcgtggaagtggttctggccgaccctaaccactggcgcttccaggggggcaagtgggtgacctgcggcaaagcggacaataacatgcagggcaataagatgtacgttcacccagaatctcctaacactggctcccactggatgaggcaggagatttcctttgggaagttaaaactcaccaataacaaaggtgcaaacaacaacaacacacagatgatagtgttgcagtctctgcacaaataccaaccgaggctgcacatcgtggaagtgacagaggacggtgtggaggacttgaatgaaccttccaagactcagaccttcaccttctcagagacacagttcatcgctgtgacggcctaccaaaacacggatatcacccagctaaagatcgaccataaccccttcgccaaaggcttccgggacaactacgattccatgtacacggcttcagaaaatgacaggttaactccatctcccacggattcccctagatcccatcagattgtccctggaggtcggtacggcgttcaaaacttcttcccggagccctttgtcaacactttgcctcaagcccgatattataacggtgagagaaccgtgccacagaccaacggcctcctctcaccccaacagagcgaagaggtggccaaccctccccagcggtggcttgtcacgcctgtccagcaacctgtgaccaacaagctagacatcggttcttatgaatctgaatatacttccagtaccttgctcccatatggtattaagtccttgcccctgcagacatcccatgccctggggtattaccctgacccgaccttccctgctatggcagggtggggaggccgtggcgcttatcagaggaagatggcagctggactaccatggacatccagaatgagcccacctgtcttcccagaagatcagcttgccaaggaaaaagttaaagaagagattagttcctcctggatagagactcccccctccatcaagtctctagactccagcgactccggggtgtacaacagcgcttgcaagagaaagcgcctgtctcccagcacccccagcaatggaaactcgccccccataaagtgtgaggacattaacactgaagagtacagtaaagacacctccaaaggcatgggggcttattatgctttttacacaagtcccggcggcggctcaggcggcgggccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatga

SEQ ID NO: 5 - DNA, 2325 bp, Artificial
  nucleic acid construct/primer sequence
atggaggtgactgcggaccagccgcgctgggtgagccaccatcaccccgcggtcctcaacggtcagcacccagacacgcaccacccgggcctcggccattcgtacatggaagctcagtatccgctgacggaagaggtggacgtactttttaacatcgatggtcaaggcaaccacgtcccgtcctactacggaaactccgtcagggctacggtgcagaggtatcctccgacccaccacgggagccaggtatgccgcccgcctctgctgcacggatctctgccctggctggatggcggcaaagccctgagcagccaccacaccgcctcgccctggaacctcagccccttctccaagacgtccatccaccacggctctccggggcctctgtccgtttaccctccggcttcatcctcttctctggcggtcggccactccagtcctcatctcttcaccttcccgcccaccccgccgaaagacgtctccccagacccgtcgctgtccaccccgggatccgccgggtcggccaggcaagatgagaaagagtgcctcaagtatcaggtgcagctgccagatagcatgaagctggagacgtctcactctcgaggcagcatgaccaccctgggtggggcctcatcctcagcccaccaccccattaccacctatccgccctatgtgcccgagtacagctctggactcttcccacccagcagcctgctgggaggatcccctaccgggttcggatgtaagtcgaggcccaaggcacgatccagcacagaaggcagggagtgtgtgaactgcggggcaacctctaccccactgtggcggcgagatggtaccgggcactacctttgcaatgcctgcggactctaccataaaatgaatgggcagaaccggccccttatcaagcccaagcgaaggctgtcggcagcaaggagagcagggacatcctgcgcgaactgtcagaccaccaccaccaccctctggaggaggaacgctaatggggacccggtctgcaatgcctgtgggctgtactaccagcttcataatattaacagacccctgactatgaagaaagaaggcatccagacccgaaaccggaagatgtctagcaaatcgaaaaagtgcaaaaaggtgcatgacgcgctggaggacttccccaagagcagctccttcaacccggccgctctctccagacacatgtcatccctgagccacatctctcccttcagccactccagccacatgctgaccacaccgacgcccatgcatccgccctccggcctctccttcggacctcaccacccttccagcatggtcaccgccatgggtggcggcggctcaggcggcgggccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatga

SEQ ID NO: 6 - DNA, 2544 bp, Artificial
  nucleic acid construct/primer sequence
atggacagggccccacagagacaccaccggacatctcgggagctgctggctgcaaagaagacccacacctcacaaattgaagtgatcccttgcaagatctgtggggacaagtcatctgggatccactacggggttatcacctgtgaggggtgcaagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatgctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacatagagtacctggtacagaatgtctgcaagtccttccgagagacatgccagctgcgactggaggaccttctacggcagcgcaccaacctcttttcacgggaggaggtgaccagctaccagaggaagtcaatgtgggagatgtgggagcgctgtgcccaccacctcactgaggccattcagtatgtggtggagtttgccaagcggctttcaggcttcatggagctctgccagaatgaccagatcatactactgacagcaggagcaatggaagtcgtcctagtcagaatgtgcagggcctacaatgccaacaaccacacagtcttttttgaaggcaaatacggtggtgtggagctgtttcgagccttgggctgcagcgagctcatcagctccatatttgacttttcccacttcctcagcgccctgtgtttttctgaggatgagattgccctctacacggccctggttctcatcaatgccaaccgtcctgggctccaagagaagaggagagtggaacatctgcaatacaatttggaactggctttccatcatcatctctgcaagactcatcgacaaggcctcctagccaagctgccacccaaaggaaaactccggagcctgtgcagccaacatgtggaaaagctgcagatcttccagcacctccaccccatcgtggtccaagccgccttcccgccactctataaggaactcttcagcactgatgttgaatcccctgaggggctgtcaaagggcggcggctcaggcggcgggccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatga

SEQ ID NO: 7 - DNA, 2586 bp, Artificial
  nucleic acid construct/primer sequence
atgggcatcgtggagccgggctgcggagacatgctgaccggcaccgagccgatgccgagtgacgagggccgggggcccggagcggaccaacagcatcgtttcttctatcccgagccgggcgcacaggacccgaccgatcgccgcgcaggtagcagcctggggacgccctactctgggggcgccctggtgcctgccgcgccgggtcgcttccttggatccttcgcctacccgccccgggctcaggtggctggctttcccgggcctggcgagttcttcccgccgcccgcgggtgcggagggctacccgcccgtggatggctaccctgcccctgacccgcgcgcggggctctacccagggccgcgcgaggactacgcattgcccgcggggttggaggtgtctgggaagctgagagtcgcgctcagcaaccacctgttgtggtccaagttcaaccagcaccagacagagatgatcatcactaagcaaggacggcgaatgttcccattcctgtccttcaccgtggctgggctggagcccacaagccattacaggatgtttgtggatgtggtcttggtggaccagcaccactggcggtaccagagcggcaagtgggtgcagtgtggaaaggcagaaggcagcatgccagggaaccgcttatatgtccacccagactcccccaacaccggagcccactggatgcgccaggaagtttcatttgggaagctaaagctcaccaacaacaagggggcttccaacaatgtgacccagatgatcgtcctgcagtctctccacaagtaccagccccggctgcacatcgtggaggtgaatgatggagagccagaggctgcctgcagtgcttctaacacacacgtctttactttccaagagacccagttcattgcagtgactgcctaccagaacgcagagatcactcagctgaaaatcgacaacaacccctttgccaaaggattccgggagaactttgagtccatgtacgcatctgttgatacgagtgtcccctcgccacctggacccaactgtcaactgcttgggggagaccccttctcacctcttctatccaaccagtatcctgttcccagccgtttctaccccgaccttccaggccagcccaaggatatgatctcacagccttactggctggggacacctcgggaacacagttatgaagcggagttccgagctgtgagcatgaagcccacactcctaccctctgccccggggcccactgtgccctactaccggggccaagacgtcctggcgcctggagctggttggcccgtggcccctcaatacccgcccaagatgagcccagctggctggttccggcccatgcgaactctgcccatggacccgggcctgggatcctcagaggaacagggctcctccccctcgctgtggcctgaggtcacctccctccagccggagtccagcgactcaggactaggcgaaggagacactaagaggaggaggatatccccctatccttccagtggcgacagctcctctcccgctggggccccttctccttttgataaggaaaccgaaggccagttttataattattttcccaacggcggcggctcaggcggcgggccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatga

SEQ ID NO: 8 - protein, 825 aa, Artificial
  polypeptide sequence
MRTQIEVIPCKICGDKSSGIHYGVITCEGCKGFFRRSQQCNVAYSCTRQQNCPIDRTSRNRCQHCRLQKCLALGMSRDAVKFGRMSKKQRDSLHAEVQKQLQQQQQQEQVAKTPPAGSRGADTLTYTLGLSDGQLPLGASPDLPEASACPPGLLRASGSGPPYSNTLAKTEVQGASCHLEYSPERGKAEGRDSIYSTDGQLTLGRCGLRFEETRHPELGEPEQGPDSHCIPSFCSAPEVPYASLTDIEYLVQNVCKSFRETCQLRLEDLLRQRTNLFSREEVTSYQRKSMWEMWERCAHHLTEAIQYVVEFAKRLSGFMELCQNDQIILLKAGAMEVVLVRMCRAYNANNHTVFFEGKYGGVELFRALGCSELISSIFDFSHFLSALCFSEDEIALYTALVLINANRPGLQEKRRVEHLQYNLELAFHHHLCKTHRQGLLAKLPPKGKLRSLCSQHVEKLQIFQHLHPIVVQAAFPPLYKELFSTDVESPEGLSKGGGSGGGPADDTYRYISAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATA

SEQ ID NO: 9 - DNA, 8422 bp, Artificial
  nucleic acid construct/primer sequence
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactaggactagtcatctttttttaagctcaagttttgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcggggtctttcatttggaggttccaccgagatttggagacccctgcccagggaccaccgacccccccgccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgaagctataagcttatgagaacacaaattgaagtgatcccttgcaagatctgtggggacaagtcatctgggatccactacggggttatcacctgtgaggggtgcaagggcttcttccgccgcagccagcagtgtaatgtggcctactcctgcacgcgtcagcagaactgccccattgaccgaaccagccgcaaccgatgccagcattgccgcctgcagaagtgcctggctctgggcatgtcccgagatgctgtcaagtttggccgaatgtccaagaagcagagggacagtctacatgcagaagtgcagaaacaactgcaacagcagcagcaacaggaacaagtggccaagactcctccagctgggagccgcggagcagacacacttacatacactttagggctctcagatgggcagctaccactgggcgcctcacctgacctacccgaggcctctgcttgtccccctggcctcctgagagcctcaggctctggcccaccatattccaataccttggccaaaacagaggtccagggggcctcctgccaccttgagtatagtccagaacgaggcaaagctgaaggcagagacagcatctatagcactgacggccaacttactcttggaagatgtggacttcgttttgaggaaaccaggcatcctgaacttggggaaccagaacagggtccagacagccactgcattcccagtttctgcagtgccccagaggtaccatatgcctctctgacagacatagagtacctggtacagaatgtctgcaagtccttccgagagacatgccagctgcgactggaggaccttctacggcagcgcaccaacctcttttcacgggaggaggtgaccagctaccagaggaagtcaatgtgggagatgtgggagcgctgtgcccaccacctcactgaggccattcagtatgtggtggagtttgccaagcggctttcaggcttcatggagctctgccagaatgaccagatcatactactgaaagcaggagcaatggaagtcgtcctagtcagaatgtgcagggcctacaatgccaacaaccacacagtcttttttgaaggcaaatacggtggtgtggagctgtttcgagccttgggctgcagcgagctcatcagctccatatttgacttttcccacttcctcagcgccctgtgtttttctgaggatgagattgccctctacacggccctggttctcatcaatgccaaccgtcctgggctccaagagaagaggagagtggaacatctgcaatacaatttggaactggctttccatcatcatctctgcaagactcatcgacaaggcctcctagccaagctgccacccaaaggaaaactccggagcctgtgcagccaacatgtggaaaagctgcagatcttccagcacctccaccccatcgtggtccaagccgccttccctccactctataaggaactcttcagcactgatgttgaatcccctgaggggctgtcaaagggcggcggctcaggcggcgggccggcggatgatacgtatcgctatatatctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaggcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagcttgatgaagcggccgcccctctccctcccccccccctaacgttactggccgaagccgcttggaataaggccggtgtgcgtttgtctatatgttattttccaccatattgccgtcttttggcaatgtgagggcccggaaacctggccctatcttcttgatgagcattcctaggggtctttcccctctcgccaaaggaatgcaaggtctgttgaatgtcgtgaaggaagcagttcctctggaagtttcttgaagataaacaacgtctgtagcaaccctttgcaggcagcggaaccccccacctggcgacaggtgcctctgcggccaaaagccacgtgtataagatacacctgtaaaggcggcacaaccccagtgccacgttgtgagttgggtagttgtggaaagagtcaaatggctctcctcaagcgtattcaacaaggggctgaaggatgcccagaaggtaccccattgtatgggatctgatctggggcctcggtgcatatgctttacatatgtttagtcgaggttaaaaaacgtctaggccccccgaaccacggggacgtggttttcctttgaaaaacacgatgataatatggccacaaccatgcagcttgccagcatgggctacctgcgccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcgccgtaggcaggtagttaacagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcggggtctttcagtttctcccacctacacaggtctcactggatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 10 - DNA, 24 bp, Artificial
  nucleic acid construct/primer sequence
atgcagtccatggtacccaactca

SEQ ID NO: 11 - DNA, 24 bp, Artificial
  nucleic acid construct/primer sequence
ctgcagaaacacagtgtggagcat

SEQ ID NO: 12 - DNA, 23 bp, Artificial
  nucleic acid construct/primer sequence
ttaagcagtacagccccaaaatg

SEQ ID NO: 13 - DNA, 25 bp, Artificial
  nucleic acid construct/primer sequence
caaacttgtctggaatttcaaatcc